(19) United States
(12) Patent Application Publication
(10) Pub. No.: US 2007/0258901 A1
(43) Pub. Date: Nov. 8, 2007

(54) Capsules Containing Transiently Transfected Cells, Method for Preparing Same and Uses Thereof

(76) Inventors: Ursula Boschert, Troinex (CH); Holger Heine, Oberwil (CH); Patricia De Lys, Saint Julien en Genevois (FR); Thierry Battle, Annemasse (FR)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/659,174

(22) PCT Filed: Aug. 2, 2005
(86) PCT No.: PCT/IB05/02294

§ 371 Date: Feb. 26, 2007

(30) Foreign Application Priority Data
Aug. 4, 2004 (EP) 04405494.8

(51) Int. Cl. A61K 48/00; A61K 49/00; C12N 15/09

(52) U.S. Cl. 424/9.2; 424/93.2; 435/455

(57) ABSTRACT

The invention concerns a capsule containing cells that are transiently transfected with a gene of interest and entrapped within a biocompatible polymer membrane, a method of preparing those capsules, a method for in vivo assessing an activity of the protein secreted by the gene of interest, which comprises administering to a multicellular organism the capsule and detecting an activity, a pharmaceutical composition comprising such a capsule, and the use thereof for the administration of a protein of interest to a subject.

### FIELD OF THE INVENTION

The present invention relates to capsules containing cells that are transiently transfected with a gene of interest and entrapped within a biocompatible polymer membrane, a method for preparing those capsules and a method using the latter for assessment of in vivo activities or functions of the protein expressed and secreted by those cells.

The invention further relates to formulations, compositions and methods that can be used for the delivery of an antigen/immunogen and/or an adjuvant for immunization and vaccination. More particularly, the invention relates to capsules containing cells that are transiently transfected with a gene of interest for more efficient and effective immunization or vaccination.

### BACKGROUND OF THE INVENTION

In vivo evaluation of protein function is a key step in the development of a new therapeutics in particular the evaluation of new soluble proteins or soluble proteins with unknown function in order to develop new protein therapeutics. Such in vivo evaluation or testing requires substantial amounts of the purified protein, which especially early in drug development are difficult to obtain. To shortcut this process, direct gene expression in vivo is an interesting alternative.

In vivo electroporation of plasmid DNA is a promising method for direct gene expression in vivo because of its low cost and its safety in utilization (Davis H. L. et al. 1999 Hum. Gen. Ther. 4, 151-159). When used under appropriate conditions, DNA electroporation of skeletal muscle fibers offers many particular advantages, such as very efficient cellular uptake of DNA and long-term transgenic expression, but the major advantage of electroporated muscle lies in its potential for the production and secretion of bioactive proteins in the bloodstream, allowing the local production of recombinant protein that exerts its effect on remote targets (Feewell J. G. et al. 2001 Mol. Ther. 3, 574-583; Kreiss et al. 1999 J. Gene Med. 1, 245-250; Aihara H et al. 1998 Nat. Biotechnol. 16, 867-870; Li S. et al. 2001 Gene Ther, 8, 400-407).

Another method for direct gene expression in vivo is the hydrodynamic delivery of DNA. It relies on the rapid intravenous injection of a large aqueous volume of naked DNA into the liver. When applied to mice and rats, it is a safe and efficient means of introducing high levels of gene expression in liver but also in other organs like kidneys, lungs and hearts but at lower level (Liu F. et al. 1999 Gene Ther 6(7), 1258-1266; Yang J. et al. 2001 Hepatology, 33(4), 848-859; Maruyama H. et al. 2002 J. Gene. Med. 4(3), 333-341; Hodges B. L. et al. 2003 Expert Opin. Biol. Ther, 3(6), 911-918).

In both electroporation and hydrodynamic gene delivery methods there is little possibility to impact on the secretion level of the transgene or the post-translational modification of the produced protein.

A third method suitable for evaluation of in vivo protein function is the administration of microencapsulated cells of a transgenic cell line, which produce in situ the desired gene product to an experimental animal such as a mouse or a rat. In this approach cells are encapsulated within a matrix, which isolates them physically from the host immune system. A perm-selective membrane is used that allows the passage of nutrients, external stimuli and the therapeutic protein, but is impermeable to the immune components that are responsible for graft rejection, and thus prolongs graft survival (Lim F. et al. 1980 Science, 210, 908-910). Such a method has been developed for gene therapy (Hortelano G. et al. 1996 Blood, 87(12), 5095-5103; Visted T. et al 2001 Neuro-Oncology 3, 201-210), for treatment of insulin-dependent diabetes mellitus (Sun Y. et al. 1996 J. Clin. Invest 98, 1417-1422) and for anti-cancer therapy (Read T-A. et al. 1999 Int. J. Devl. Neuroscience, 17 (5-6), 653-663). Full control of the level of transgene expression and post-translational modifications is possible via the choice of the encapsulated cell line, the cell density in capsules and the possibility to include regulatory elements on the expression cassette. Several biocompatible polymers can be used for the cell immobilization depending on the properties of the therapeutic protein to be expressed. Alginate-poly-L-Lysine-Alginate (APA) capsules are most frequently used (cf. Lim F. et al. 1980 Science, 210, 908-910; Hortelano G. et al. 1996 Blood, 87(12), 5095-5103; Visted T. et al 2001 Neuro-Oncology 3, 201-210; Sun Y. et al. 1996 J. Clin. Invest 98, 1417-1422; Read T-A. et al. 1999 Int. J. Devl. Neuroscience, 17(5-6), 653-663; and Strand B. L. et al. 2002 J. Microencapsulation 19(5), 615-630) but other polymers like poly(hyroxyethyl methacrylate-co-methyl methacrylate) (HEMA-MMA) can be used as well (Lahooti S. et al. 2000 Biomaterials, 21, 987-995). The encapsulation matrix can be either completely solid or contain a liquid core with only a membrane separating the entrapped cells from the environment.

Savelkoul et al. (Savelkoul Huub F. J. et al 1994 Journal of Immunological Methods 170 pp. 185-196) describes the preparation and encapsulation of cells from stably transfected monkey CV1 or CHO-Ki cell lines producing Il-4 or IL-5 (cf. page 186 2.3 and 2.4), and intraperitoneal (i.p.) or subcutaneous (s.c.) injection of said encapsulated cells into mice.

WO 93/00439 discloses the encapsulation of cells of genetically engineered cell lines that produce an active factor or augmentary substance, in particular the rat N8-21 NGF releasing cell line (Example 1).

In all examples described in the prior art supra the encapsulated cells came where stably transfected cells.

The generation of stably transfected cell lines is laborious and time-consuming since it requires the transfected gene to be integrated into the genome of the cell. This is achieved by prolonged cultivation of the transfected cells in a selection medium followed by serial dilutions of the cultivated cells in order to isolate cell clones that are stably transfected and produce the protein of interest in sufficient amounts (ref. to Maniatis et al.) Stably transfected cells are generally selected for high production of the protein of interest. Although stably transfected cells offer the advantage of high and prolonged expression of the transfected gene (up to several months).

The above methods, however, are not suitable for applications requiring high throughput and/or speed such as the evaluation of an in vivo activity of multiple proteins.

The problem addressed by the present invention is to provide a method for protein expression in vivo, which is suitable for high throughput and can be performed within a short time (e.g. one or two or more weeks) and to provide means for evaluation of in vivo activities of proteins, which do not have the drawbacks of the prior art methods.

The above problem is solved by the invention as defined in the claims.

Activation of the immune system of vertebrates is an important mechanism for protecting animals against pathogens and malignant tumors. The immune system consists of many interacting components including the humoral and cellular branches. Humoral immunity involves antibodies that directly bind to antigens. Antibody molecules as the effectors of humoral immunity are secreted by B lymphocytes. Cellular immunity involves specialized cytotoxic T lymphocytes (CTLs), which recognize and kill other cells that produce non-self antigens. CTLs respond to degraded peptide fragments that appear on the surface of the target cell bound to MHC (major histocompatibility complex) class I molecules. It is understood that proteins produced within the cell are continually degraded to peptides as part of cellular metabolism. These fragments are bound to the MHC molecules and are transported to the cell surface. Thus the cellular immune system is constantly monitoring the spectra of proteins produced in all cells in the body and is poised to eliminate any cells producing non-self antigens.

Vaccination is the process of priming an animal for responding to an antigen/immunogen. The antigen can be administered as purified protein, protein contained in killed/attenuated pathogens, or as a gene that then expresses the antigen in host cells (genetic immunization). The process involves T and B lymphocytes, other types of lymphoid cells, as well as specialized antigen presenting cells (APCs) that can process the antigen and display it in a form that can activate the immune system.

The efficacy of a vaccine is measured by the extent of protection against a later challenge by a tumor or a pathogen. Effective vaccines are immunogens that can induce high titer and long-lasting protective immunity for targeted intervention against diseases after a minimum number of inoculations.

The number of successful approaches to vaccine development is almost as broad as the number of infectious agents. As technology has developed, it has become possible to define at the molecular level the nature of the protective antigen/immunogen. In recent years, acellular vaccines have become the method of choice for vaccine development because they can be administered with subunits from a variety of pathogens (i.e. multicomponent vaccines) and they have the potential for reduced numbers of adverse reactions. Subunit vaccines are composed of defined purified protective antigens from pathogenic microorganisms. Although there have been a few stunning successes, a small number of subunit vaccines are currently in use. Perhaps the most daunting reason impeding the use of subunit vaccines is the problem of antigen delivery. For optimal antigen delivery, the antigen needs to be delivered to the antigen presenting cells in its biological context, or the antigen risks to be readily recognized and taken up by phagocytes. Most antigens possess three dimensional structures that are important for parasite-host cell interactions and many of these structures are lost during antigen purification.

One approach taken to circumvent most of the problems associated with subunit vaccine production is the development of live recombinant vaccine vehicles, based on attenuated viruses and bacteria that have been genetically engineered to express protective antigens in vivo (i.e. recombinant forms of vaccinia virus, adenovirus, Salmonella and mycobacterium tuberculosis typhus bonivur var. Bacille-Calmette-Guerin or BCG) (see Snapper, S. B. et al., Proc. Natl. Acad. Sci USA 85:6987-6991, 1988; Jackett, P. S. et al., J. Clin. Micro. 26:2313-2318, 1988; Lamb, J. R. et al., Rev. Infect. Dis. 11:S443-S447, 1989; Shinnick, T. M. et al., Infect. Immun. 56:446-451, 1988).

Vaccination using live bacteria has been studied, and often utilizes a live bacteria strain in which a mutation has been induced to knock-out the lethal gene. Live vaccines present advantages in that the antigen is expressed in the context of an innately immunogenic form; the live delivery system replicates and persists in the host, re-stimulating the host immune system and obviating the need for multiple doses; live vector systems eliminate the need to purify the antigen, and are less expensive to produce; and live vectors can be designed to deliver multiple antigens, reducing the number of times an individual must be vaccinated. However, this method requires extreme safety precautions to ensure that a further mutation does not occur that would allow the bacterium to return to virulence. A more reliable method is to utilize a weakened bacterium to express a protein to which the host can then produce antibodies against. Often, a bacterial vector is studied for oral administration of a vaccine; for example, Salmonella-based vaccines are being researched for oral administration to protect against HIV, Lyme disease, and Epstein-Barr virus.

Baculovirus, yeast and tissue culture cells have also been studied for use in the production of vaccines. Examples are shown in U.S. Pat. No. 6,287,759 where baculovirus is employed to produce a protein used in a vaccine against Hepatitis E; U.S. Pat. No. 6,290,962 wherein yeast is used as a vector to produce a Helicobacter polypeptide for use in a vaccine; and U.S. Pat. No. 6,254,873 wherein vertebrate tissue culture cells are used to propagate purified inactivated dengue virus for use in a vaccine. In all of these examples, the vectors were used to produce a protein of interest, would then be purified and used in the vaccine.

Genetic immunization is another approach to elicit immune responses against specific proteins by expressing genes encoding the proteins in an animal's own cells. The substantial antigen amplification and immune stimulation resulting from prolonged antigen presentation in vivo can induce a solid immunity against the antigen. Genetic immunization simplifies the vaccination protocol to produce immune responses against particular proteins because the difficult steps of protein purification and combination with adjuvant, both routinely required for vaccine development, are eliminated. Since genetic immunization does not require the isolation of proteins, it is especially valuable for proteins that may lose conformational epitopes when purified biochemically. Genetic vaccines may also be delivered in combination without eliciting interference or affecting efficacy (Tang et al., 1992; Barry et al., 1995), which may simplify the vaccination scheme against multiple antigens.

Vaccines are often augmented through the use of adjuvants. Vaccine adjuvants are useful for improving an immune response obtained with any particular antigen in a vaccine composition. Adjuvants are used to increase the amount of antibody and effector T cells produced and to reduce the quantity of antigen and the frequency of injection. Although some antigens are administered in vaccines without an adjuvant, there are many antigens that lack sufficient immunogenicity to stimulate a useful immune response in the absence of an effective adjuvant. Adjuvants also improve the immune response from “self-sufficient” antigens, in that the immune response obtained can be increased or the amount of antigen administered can be reduced.

For a comprehensive review on adjuvants and delivery systems for molecular vaccination see Sheikh NA, Al-Shamisi M, Morrow W J W; Delivery systems for molecular vaccination; Current Opinion in Molecular Therapeutics 2000,2:1(37-54).

In order to develop vaccines against pathogens or tumors that have been recalcitrant to vaccine development, and/or to overcome the failings of commercially available vaccines due to underutilization, new methods of antigen/immunogen delivery must be developed which will allow for fewer immunizations, and/or fewer side effects of the vaccine.

A new pharmaceutical composition comprising an antigen/immunogen and vaccine delivery methods are described in this application wherein the antigen/immunogen is produced in vivo following the delivery of encapsulated cells that are transiently transfected with one or more genes coding for a suitable antigen and/or an adjuvant. This method overcomes the disadvantages of other vaccines and methods for vaccination described above such as the need of cumbersome purification of the antigen/immunogen associated with protein vaccines or the low efficiency due to low in vivo production of antigen/immunogen associated with genetic immunization.

### SUMMARY OF THE INVENTION

The invention relates to a capsule containing cells that are transiently transfected with a gene of interest and entrapped within a biocompatible polymer membrane.

In another aspect the invention provides said capsule wherein the cells are animal cells, preferably mammalian cells.

In a further aspect said capsule is a capsule, wherein the gene of interest is fused to a signal sequence for secretion of the protein.

In yet another aspect of the invention the capsule harbors the gene of interest inserted into an expression cassette.

In a further aspect the capsule harbors the gene of interest inserted into a plasmid.

In yet another aspect of the invention said capsule comprises the biocompatible polymer alginate-poly-L-lysine-alginate (APA).

In a further aspect the capsule comprises a biocompatible polymer membrane that has pores with a cut-off size of 90 to 30, preferably 80 to 60 kDa.

In even a further aspect of the invention the capsule has a mean diameter of 100 to 1500 μm, preferably 250 to 600 μm, in particular 440 to 530 μm.

In another aspect of the invention the capsule has been maintained under low-shear microgravity conditions.

The invention further provides a method of preparing a capsule comprising the steps of transiently transfecting cells with a gene of interest and encapsulating the transiently transfected cells.

The invention further provides a method of preparing a capsule comprising the further step of maintaining the capsules under low-shear microgravity gravity conditions.

The invention further provides a method for assessing an in vivo activity of the protein encoded by a gene of interest and expressed and secreted by a transiently transfected and encapsulated cell, which comprises administering the capsule mentioned supra to a multicellular organism and detecting an activity of said protein in said multicellular organism.

The invention further provides a method for assessing an in vivo activity of the protein encoded by a gene of interest and expressed and secreted by a transiently transfected and encapsulated cell, which comprises administering the capsule mentioned supra to a multicellular organism wherein the multicellular organism is a mammal.

The invention further provides a method for assessing an in vivo activity of the protein encoded by a gene of interest and expressed and secreted by a transiently transfected and encapsulated cell, which comprises administering the capsule mentioned supra to a mammal, wherein the mammal is selected from the group consisting of mouse, rats, dogs, goats, sheep, cows and monkeys.

The invention further provides a method for assessing an in vivo activity of the protein encoded by a gene of interest and expressed and secreted by a transiently transfected and encapsulated cell, which comprises administering the capsule mentioned supra to a multicellular organism, wherein administration of the capsule is performed by i.p. injection.

The invention further provides the use of the capsule or the methods mentioned above in an animal where the activity to be detected occurs rapidly (e.g. within hours, or one or two or more days after the injection) and is completed within a period of a few days after administration of the active substance.

The invention further provides the use of the capsule or the methods mentioned above in an animal where the activity to be detected occurs rapidly (e.g. within hours, or one or two or more days after the injection) and is completed within a period of a few days after administration of active substance, where the animal is a mouse with Concanavalin A (ConA) induced liver toxicity.

The invention further provides a pharmaceutical composition comprising the capsule containing cells that are transiently transfected with a gene of interest and entrapped within a biocompatible polymer membrane.

The invention further provides a pharmaceutical composition as described above, wherein the cells are transfected with a gene coding for an antigen/immunogen and/or an adjuvant.

The invention further provides a pharmaceutical composition as described, wherein the antigen/immunogen is a bacterial, viral, fungal, parasitic or tumor antigen.

The invention further provides a pharmaceutical composition as described, wherein the capsule comprises at least two types of cells each transfected with a gene coding for (an) antigen(s) and/or adjuvant(s), and wherein the two genes are not identical.

The invention further provides the use of the capsule containing cells that are transiently transfected with a gene of interest and entrapped within a biocompatible polymer membrane for the administration of a protein of interest to a subject.

The invention further provides the use of the capsule containing cells that are transiently transfected with a gene of interest and entrapped within a biocompatible polymer membrane for the administration of a protein of interest to a subject, wherein the protein is an antigen for immunization or vaccination.

The invention further provides the use of the capsules containing cells that are transiently transfected with a gene of interest and entrapped within a biocompatible polymer membrane for the administration of a protein of interest to a subject, wherein the subject is selected from the group consisting of humans, animals kept for research purposes including rodents, dogs, pigs and monkeys, livestock and companion animals including dogs and cats.

The invention further provides a kit comprising a capsule or a pharmaceutical composition as described herein and means for the administration of said capsule or composition to a subject.

### BRIEF DESCRIPTION OF THE FIGURES

FIG. 1 represents the full coding sequence and the translated sequence of human IL-6 (hIL-6). (Seq. Id. No. 1 and 2)

FIG. 2A represents the restriction map and FIG. 2B the complete nucleotide sequence, of the mammalian cell expression vector pEAK12d. (Seq. Id. No. 11)

FIG. 3 represents the restriction map of the mammalian cell expression vector pEAK12d-IL-6-6HIS.

FIG. 4 represent histograms showing the TNFα, ASAT and ALAT levels measured in blood samples of ConA treated mice that were injected with capsules containing non-tranfected HEK293-EBNA cells, and control mice.

FIG. 5A represents a graph of the hIL-6 levels measured in blood of mice during 12 consecutive days after i.p. or s.c. injection of capsules containing HEK293-EBNA cells that were transiently transfected with the cDNA coding for hIL-6 HIS.

FIGS. 5B-D represent histograms showing the TNFα, ASAT and ALAT levels measured in blood samples of ConA treated mice that were injected with capsules containing HEK293-EBNA cells that were transiently transfected with the cDNA coding for hIL-6 HIS, and control mice.

FIG. 6 represents a histogram showing the hIL-6 levels measured in blood of mice during 3 consecutive days after i.p. injection of 700, 350 and 100 μl, respectively, of capsules containing HEK293-EBNA cells that were transiently transfected with the cDNA coding for hIL-6 HIS.

FIGS. 7A-C represent histograms showing the TNFα, ASAT and ALAT levels measured in blood samples of ConA treated mice that were injected with 700, 350 and 100 μl, respectively, of capsules containing HEK293-EBNA cells that were transiently transfected with the cDNA for hIL-6-6HIS, and control mice.

FIGS. 8A and 8B represent the sequence of the gene for hepaCAM (Seq. Id. No. 7) and the sequence of the mature protein (Seq. Id. No. 8), respectively.

FIGS. 8C and 8D are histograms showing the TNFα levels in the mice model of LPS-induced TNFα release after i.p. or s.c. injection (respectively) of untransfected encapsulated HEK293-EBNA cells and different quantities of encapsulated HEK293-EBNA cells that are transiently transfected with a gene encoding hepaCAM.

FIGS. 9A and 9B represent the sequence of the gene encoding INSP114-SV2 (Seq. Id. No. 9) and the sequence of the mature protein (Seq. Id. No. 10), respectively.

FIG. 9C is a histogram showing the TNFα levels in the mice model of LPS-induced TNFα release after i.p. injection of untransfected encapsulated HEK293-EBNA cells and encapsulated HEK293-EBNA cells that are transiently transfected with a gene for INSP114-SV2.

FIGS. 10A and 10B represent the sequence of the gene encoding human erythropoietin (EPO) (Seq. Id. No. 5) and the sequence of the mature protein (Seq. Id. No. 6), respectively.

FIG. 10C is a graph showing the concentration of EPO in the blood of mice measured by ELISA on day 0 to day 15 after i.p. or s.c. injection of encapsulated HEK293-EBNA cells transiently transfected with the gene encoding EPO, encapsulated HEK293-EBNA cells stably transfected with the gene encoding EPO and encapsulated control HEK293-EBNA cells (non-transfected).

FIG. 10D is a graph showing the hematocrit (hematocrit (Ht) or packed cell volume (PCV) is the proportion of blood volume that is occupied by red blood cells) in mice from day 0 to day 10 after i.p. or s.c. injection of HEK293-EBNA cells transiently transfected with the gene encoding EPO and HEK293-EBNA cells stably transfected with the gene encoding EPO.

FIGS. 11A and 11B represent the sequence of the gene for mouse IL-18 binding protein (m-IL18BP) (Seq. Id. No. 3) and the sequence of the mature protein (Seq. Id. No. 4), respectively.

FIG. 11C is a histogram showing the concentration of m-IL18BP in blood of mice measured by ELISA on day 0 to day 8 after i.p. injection of encapsulated HEK293-EBNA cells that are transiently transfected with a gene encoding m-IL18BP.

### DESCRIPTION OF THE INVENTION

The invention provides capsules containing cells that are transiently transfected with a gene of interest and entrapped within a biocompatible polymer membrane.

The term “capsules” or “capsule”, which is used interchangeably refers to an encapsulation matrix that contains cells with only a biocompatible polymer membrane separating the cells from the environment.

The term “capsules” or “capsule” further refers to one or more cells that are enclosed or entrapped in a biocompatible polymer membrane.

The term “transiently transfected cell(s)” or “cell(s) transiently transfected with a gene of interest” refers to (a) cell(s), into which a gene of interest has been introduced by methods known in the art (ref. Maniatis et al.), and which express the protein encoded by the gene of interest for less or equal than 14 days, and preferentially for less or equal than 10 days starting from the day of transfection.

The term “transiently transfected” or “transiently transfected with a gene of interest” further refers to the introduction of a gene of interest into a pre-selected cell by methods known in the art (ref. Maniatis et al.), whereby said cell contains the gene of interest but the latter is not integrated into the genome of said cell, or located on an episomal vector that is able to replicate within the cell.

Transiently transfected cells are preferably obtained without applying any selection pressure by a selection medium, when cultivating the cells after transfection.

Selection media used in cell culture are well known in the art. Several different drug selection markers are commonly used for long-term transfection studies. For example, cells transfected with recombinant vectors containing the bacterial gene for aminoglycoside phosphotransferase can be selected for stable transfection in the presence of the drug G-418 (Southern, P. J. and Berg, P. (1982) Transformation of mammalian cells to antibiotic resistance with a bacterial gene under control of the SV40 early region promoter, J. Mol. Appl. Gen. 1, 327). Similarly, expression of the gene for hygromycin B phosphotransferase from the transfected vector will confer resistance to the drug hygromycin B (Blochlinger, K. and Diggelmann, H. (1984) Hygromycin B phosphotransferase as a selectable marker for DNA transfer experiments with higher eucaryotic cells, Mol. Cell. Biol. 4, 2929).

An alternative strategy is to use a vector carrying an essential gene that is defective in a given cell line. For example, CHO cells deficient in expression of the dihydrofolate reductase (DHFR) gene survive only in the presence of added nucleosides. However, these cells, when stably transfected with DNA expressing the DHFR gene, will synthesize the required nucleosides (Stark, G. R. and Wahl, G. M. (1984) Gene amplification. Ann. Rev. Biochem. 53, 447.). An additional advantage of using DHFR as a marker is that gene amplification of DHFR and associated transfected DNA occurs when cells are exposed to increasing doses of methotrexate (Schimke, R. T. (1988) Gene amplification in cultured cells. J. Biol. Chem. 263, 5989).

The term “stably transfected” or “stably transfected with a gene of interest” or “stable transfection” refers to the introduction of a gene of interest into a pre-selected cell by methods known in the art (ref. Maniatis et al.), whereby said gene of interest is integrated into the genome of said cell, or located on an episomal vector and replicated within the cell.

The term “gene of interest” refers to genomic DNA, cDNA, synthetic DNA, RNA and other polynucleotides or analogues thereof that code for a protein of interest, i.e. a protein having interesting activities or a protein of which the properties are of interest, e.g. are to be assessed.

Surprisingly it has been found by the present inventors that encapsulated cells that are transiently transfected with a gene of interest can be administered (e.g. injected) to a multicellular organism and produce sufficient amounts of the protein encoded by the gene of interest in vivo, such that an activity or effect of said protein in vivo can be detected.

Further surprisingly said encapsulated cells can be administered to the multicellular organism at same day of transfection, or also one or more days thereafter, and an activity or effect of said protein in vivo can be detected.

The invention thus provides a novel tool for the analysis or assessment of an in vivo activity of a protein encoded by a gene of interest within not more than 14 days starting from the administration, preferentially not more than 10 days and even more preferred not more than 5 days.

In a first aspect the invention provides novel capsules containing cells that are transiently transfected with a gene of interest and entrapped within a biocompatible polymer membrane.

The cells may be plant or animal cells. Preferably they are animal cells, in particular mammalian cells including hybridomas. Particularly preferred cells are COS cells, BHK cells, VERO cells, CHO cells, rCHO-tPA cells, rCHO-Hep B Surface Antigen cells, HEK293 cells, rHEK293 cells; examples of hybridomas that can be cultivated according to the present invention include, e.g., DA4.4 cells, 123A cells, 127A cells, GAMMA cells and 67-9-B cells; examples of insect cells that can be cultivated according to the present invention include, e.g., lepidopteran cells, Tn-368 cells, SF9 cells, rSF9 cells and Hi-5 cells (see, e.g., Ikonomou L et al. November-December 2002, 18(6), 1345-1355); examples of non-mammalian cells that can be cultivated according to the present invention include, e.g., brown bullhead cell lines (see, e.g., Buck CD et al. February 1985, 10(2), 171-84).

The gene of interest as defined supra is expressed and a protein secreted from the cells. Preferentially the protein is secreted as mature protein. The protein when expressed preferentially comprises a signal peptide for protein secretion. If a gene under study does not possess a sequence coding for the signal peptide of the protein, such a sequence, e.g. a signal sequence, a pre-sequence and/or a pro-sequence, may be fused to that gene sequence, so as to make the gene of interest apt to be expressed, exported and secreted as protein.

The gene of interest is generally inserted into an expression cassette comprising a promoter and regulating signals, which is usually part of a vector suitable for transient transfection of cells.

Suitable vectors are plasmids or episomal vectors.

Examples of suitable plasmids for transient transfection of cells are pCEP4 (Invitrogen, USA), the pEAK vector family (Edge Biosystems, USA), or pCDNA3.1 (Invitrogen, USA).

Transfection of the cells with a vector carrying the gene of interest may be performed by any transfection method, notably the polyamine method, the polyethyleneimine method (O. Boussif et al. 1995 Proc. Natl. Acad. Sci. USA 92, pp. 7297-7301), calcium-phosphate co-precipitation or lipofection. The polyamin method is preferred since it yields more than 90% of transfected cells. The shotgun approach or electroporation are not preferred for this application since they entail a high rate of cellular death, which would yield to encapsulation of a large proportion of dead cells and hence reduced productivity of the capsules.

The biocompatible polymer may be selected among the biocompatible polymers known in the art for encapsulating cells of stably expressing cell lines, notably alginates, in particular sodium or potassium alginate, and alginate-poly-L-lysine-alginate (APA), and other polymers like poly(hyroxyethyl methacrylate-co-methyl methacrylate) (HEMA-MMA). A preferred biocompatible polymer is APA.

The polymer is engineered to make the membrane perm-selective, i.e. permeable to nutrients, oxygen, external stimuli and the secreted protein, but not to the immune components that are responsible for graft rejection. Such a perm-selective membrane has usually a porosity with a cut-off of 90 to 30, preferably 90 to 50 or 80 to 50 kDa, and even more preferably from 80 to 60 kDa. The cut-off defines the size of molecules that can diffuse through the perm-selective membrane.

Generally the capsules have a diameter of 100 to 1500 μm, preferably 250 to 600 μm, in particular 440 to 530 μm.

The invention also relates to a method for preparing the above capsules comprising the steps of transiently transfecting the cells with the gene of interest and encapsulating the transiently transfected cells.

After encapsulation, the cells continue to grow in the capsules and diffuse the mature protein secreted by the gene of interest into the medium in which the capsules are maintained.

Preferably the capsules are maintained under low-shear microgravity conditions, which lower the in vitro mature protein diffusion and avoid any disruption of the capsules for mechanical reasons.

All steps of the preparation of the capsules of the invention can be performed in a short time, usually within one or two days.

For storage up to several months, capsules containing cells can be frozen at −80° C. or in the vapor or liquid phase of liquid nitrogen (−196° C). This cryopreservation allows for large quantities of capsules to be produced and subsequent usage of aliquots thereof for different applications (e.g. in a kit). After resuscitation the cells restart to grow within the capsules and continue to produce the protein of interest during 3-10 days.

In a further aspect the invention concerns a method for analyzing or assessing an in vivo activity of the protein encoded by the gene of interest and expressed and secreted by the transiently transfected cell, which comprises administering capsules as defined above to a multicellular organism and detecting an activity of said protein. The activity can be a local or a systemic activity.

The proteins that may be produced according to a method of the present invention can be any protein of interest including, e.g., chorionic gonadotropin, follicle-stimulating hormone, lutropin-choriogonadotropic hormone, thyroid stimulating hormone, human growth hormone, interferons (e.g., interferon beta-1a, interferon beta-1b), interferon receptors (e.g., interferon gamma receptor), TNF receptors p55 and p75, TACI-Fc fusion proteins, interleukins (e.g., interleukin-1, interleukin-2, interleukin-3, interleukin-4, interleukin-5, interleukin-6, interleukin-8, interleukin-10, interleukin-11, interleukin-12), interleukin binding proteins (e.g., interleukin-18 binding protein), growth factors (e.g. erythropoietin, granulocyte colony stimulating factor, granulocyte-macrophage colony-stimulating factor, platelet-derived growth factor, acidic and basic fibroblast growth factor, keratinocyte growth factor, glial cell line-derived neurotrophic factor), pituitary peptide hormones, menopausal gonadotropin, insulin-like growth factors (e.g., somatomedin-C), thrombomodulin, insulin, Factor VIII, somatropin, bone morphogenetic protein-2, hirudin, epoietin, recombinant LFA-3/IgG1 fusion protein, glucocerebrosidase, and muteins, fragments, soluble forms, functional derivatives, fusion proteins thereof.

The multicellular organism may be a plant or an animal. An animal of particular interest is a mammal that is commonly used in pharmaceutical research, such as a mouse, rat, dog, goat, sheep, pig, cow or monkey.

Particularly interesting mammals are rodents such as mice or rats, preferred are mice, e.g. of strain C57/BL6.

The activity detected may concern any parameter such as e.g. body temperature, tissue or body fluid color, concentration of a given substance in a tissue or a body fluid such as blood, serum, plasma, feces, sputum, synovial fluid, cerebrospinal fluid or urine.

The capsules may be administered by intramuscular (i.m.), intradermal, subcutaneous (s.c.), or intraperitoneal (i.p.) injection. A preferred mode of administration is i.p. injection. Another preferred mode is s.c. injection.

The capsules are stable under in vivo conditions and do not break or release the entrapped cells.

The capsules release in vivo the mature protein as long as transient expression takes place, i.e. for a period generally of 3 to 14 days; most of the release usually takes place from day 1 to day 4, 5 or 6, after administration of the capsules (day 0). The period of release of a high amount of the mature protein, usually from 3 to 5 days, is generally sufficient for inducing a local and/or a systemic activity to be detected. If need be, e.g. for detecting a chronic effect, two or more administrations of the capsules can be performed at different times such as to maintain release of a high amount of the mature protein for a longer period.

The capsules of the invention thus represent useful tools in pharmaceutical research that can be used in laboratory animals, e.g. animal models of human disease.

Preferably the animal or animal model is one where the induced local and/or systemic activity to be detected occurs rapidly (i.e. within hours, or one or two or more days) and is completed within a period of a few days after administration of active substance (protein of interest).

An example of such an animal model is the mouse Concanavalin A (ConA) induced liver toxicity, which will be illustrated thereafter.

Toxic liver disease represents a worldwide health problem in humans for which pharmacological treatments have yet to be discovered. For instance active chronic hepatitis leading to liver cirrhosis is a disease state in which activated T cells progressively destroy liver parenchymal cells. ConA induced liver toxicity is one of three experimental models of T-cell dependent apoptotic and necrotic liver injury described in mice. Gal N (D-Galactosamine) sensitized mice challenged with either activating anti-CD3 monoclonal AB or with superantigen SEB develop severe apoptotic and secondary necrotic liver injury . Injection of the T-cell mitogenic plant lectin ConA to non-sensitized mice results also in hepatic apoptosis that precedes necrosis. Con A induces the release of systemic TNFα and IFNγ and various other cytokines. Transaminase release 8 hours after the insult indicates severe liver destruction.

Both TNFα and IFNγ are critical mediators of liver injury in inducing liver damage. TNFα for example is one of the first cytokines produced after ConA injection and anti TNFα antibodies confer protection against disease (Seino et al. 2001, Annals of surgery 234, 681). The later induced IFNγ seems also to be a critical mediator of liver injury since anti-IFNγ antiserum significantly protects mice, as measured by decreased levels of transaminases in the blood of ConA treated animals (Kuesters et al Gastroenteroloy 111, 462). There is a wide range of literature describing treatments conferring protection from ConA induced liver toxicity. A single administration of rIL6 completely inhibited the release of transaminases, whereas the same regime induced only 40-50% inhibition of TNF production. (Mizuhara et al. 1994, J. Exp. Med. 179, 1529-1537).

Several cell types have been shown to be involved in liver damage, CD4 T cells, macrophages and natural killer cells (Kaneko J. Exp. Med. 2000, 191, 105-114). Anti CD4 antibodies block activation of T cells and consequently liver damage (Tiegs et al. 1992, J. Clin. Invest. 90, 196-203). Pretreatment of mice with a monoclonal antibody against CD8 failed to protect, whereas deletion of macrophages prevented the induction of hepatitis.

Another example of an interesting animal model is the mice LPS (lipopolysaccharide) induced TNFα release model.

LPS is a large molecule that contains both lipid and a carbohydrate. It is a major component of the cell wall of Gram-negative bacteria. LPS acts as the protypical endotoxin and promotes the secretion of pro-inflammatory cytokines in many cell types. LPS is recognized by phagocytic cells of the innate immune system via the Toll receptor 4 (Triantafilou M. et al. Trends Immunol. June 2002; 23(6):301-4). LPS is widely used to activate macrophages or microglia in vitro or in vivo (Tobias PS et al. Immunobiology. April 1993; 187(3-5):227-32). The LPS induced TNFα release model in the mice mimics an inflammatory situation in humans.

The invention further provides a pharmaceutical composition comprising a capsule as described above where the cells are transiently transfected with one or more genes. The pharmaceutical composition optionally comprises additionally one or more pharmaceutically acceptable carriers, diluents or excipients.

The gene may encode one or more antigens/immunogens or one or more portions thereof, or one or more epitopes of interest, from a pathogen. The term antigen/immunogen refers to any protein or derivative thereof that is capable of electing an immune response in a host.

In a preferred embodiment the pathogen is selected from the group consisting of any virus, chlamydia, mycoplasma, bacteria, parasites or fungi. Viruses include the herpesviruses, orthomyxoviruses, rhinoviruses, picornaviruses, adenoviruses, paramyxoviruses, coronaviruses, rhabdoviruses, togaviruses, flaviviruses, bunyaviruses, rubella virus, reovirus, hepadna viruses and retroviruses including human immunodeficiency virus. Bacteria include mycobacteria, spirochetes, rickettsias, chlamydia, and mycoplasma. Fungi include yeasts and molds. Parasites include schistosoma spp., leishmania spp. and plasmodium spp. It is to be understood that this list does not include all potential pathogens against which a protective immune response can be generated according-to the methods herein described.

In particular the gene may encode one or more antigens/immunogens selected from the group consisting of: influenza hemagglutinin, influenza nuclear protein, influenza M2, tetanus toxin C-fragment, anthrax protective antigen, anthrax lethal factor, anthrax germination factors, rabies glycoprotein, HBV surface antigen, HIV gp120, HIV gp160, malaria CSP, malaria SSP, malaria MSP, malaria pfg, botulinum toxin A, and mycobacterium tuberculosis HSP.

In another embodiment the gene may encode an antigen/immunogen from a tumor. The tumor can be a tumor of breast, ovarian, lung, brain, stomach, gut, pancreas, bladder, prostate, bone or hematopoeitic cells or tissue or a tumor of other cells or tissue. In particular the gene may encode one or more antigens/immunogens selected from the group consisting of: HER2/neu, human carcinoembryonic antigen and prostate specific antigen.

In another embodiment the gene may encode any antigen/immunogen selected to raise antibodies against. The antibodies can be polyclonal or monoclonal. The antibody may be in particular a murine, chimeric, humanized, fully human, domain or single chain antibody. The antibody may be useful e.g. for research, diagnostic, therapeutic or other purposes.

In yet another embodiment the gene may encode an adjuvant. The adjuvant can be a co-stimulatory molecule, cytokine or chemokine. Preferred adjuvants are IL-2, IL-4, IL-6, IL-12, IL-18, GM-CSF or INF gamma.

In another embodiment the pharmaceutical composition comprises capsules (a) comprising cells that are transfected with one or more antigens/immunogens and/or adjuvants as described herein or comprising (b) groups of cells, which are transfected with a different selection of one or more antigens/immunogens and/or adjuvants as, described herein. It will be appreciated that the selection of cells in the capsules can be adapted to needs of the vaccination. This can be achieved by the appropriate selection of antigens/immunogens and adjuvants, the appropriate selection of an expression vector and an expression cassette, by the appropriate selection of the cell type or cell line expressing the gene of interest and/or the appropriate selection of the encapsulation material as described herein and in the art.

Another embodiment of the invention is a method of immunization comprising administering the capsules or the pharmaceutical composition as described herein to a subject.

Another embodiment of the invention is the use of the capsules or the pharmaceutical composition as described herein for the administration of a protein of interest to a subject.

A preferred embodiment is the use of the capsules or the pharmaceutical composition as described herein for the administration of a protein of interest to a subject, wherein the protein is an antigen/immunogen for vaccination.

A preferred embodiment is the use of the capsules or the pharmaceutical composition as described herein for the administration of a protein of interest to a subject, wherein the protein is an antigen/immunogen that has been selected as a target for an antibody. In particular the capsules are used to provoke an antibody response in a subject against a preselected antigen that is produced by the encapsulated cells.

Another embodiment of the invention is a kit comprising a capsule or a pharmaceutical composition as described herein and means for the application of said capsule or composition to the subject.

The subject can be an animal and is advantageously a vertebrate such as a mammal, bird, reptile, amphibian or fish; more advantageously a human, or a companion animal or a domesticated or food-producing or feed-producing animal or livestock or game or racing or sport animal such as a cow, a dog, a cat, a goat, a sheep or a pig or a horse, or even fowl such as turkey, ducks or chicken. In another embodiment the vertebrate is an animal kept for research purposes including but not limited to rodents (including mice, guinea pigs and rats) dogs, pigs and monkeys. In the most preferred embodiment of the invention, the vertebrate is a human.

The capsules or the pharmaceutical compositions disclosed herein are preferentially administered to the subject through a parenteral route. For example, an individual can be inoculated by intraperitoneal, intradermal, subcutaneous or intramuscular methods.

Other features and advantages of the present invention will become apparent from the examples below, which have an illustrative and not a limitative character. The examples will conveniently be read by referring to the appended figures.

### EXAMPLE 1

### Preparation of Capsules Containing Cells Transiently Transfected with DNA Coding for IL-6 and Measurement of the Blood Level of IL-6 and the Activities in the Mice Concanavalin A Model

1. Preparation of Capsules Containing Cells that are Transiently Transfected with the Gene of Interest

1.1 Cell Culture and Maintenance

Cells of Human Embryonic Kidney 293 cell line expressing the Epstein-Barr virus Nuclear Antigen (HEK293-EBNA, Invitrogen, USA) were maintained in suspension in Ex-cell VPRO serum-free medium (maintenance medium, JRH, UK) supplemented with 4 mM L-Glutamine (Invitrogen) and 1 ml/l Phenol-Red-solution (0.5% w/v in water, Phenol Red: Sigma, USA) in spinner flasks (Techne, UK).

1.2 Plasmid Preparation

Construction of Plasmids for Expression of IL-6 in HEK293-EBNA Cells.

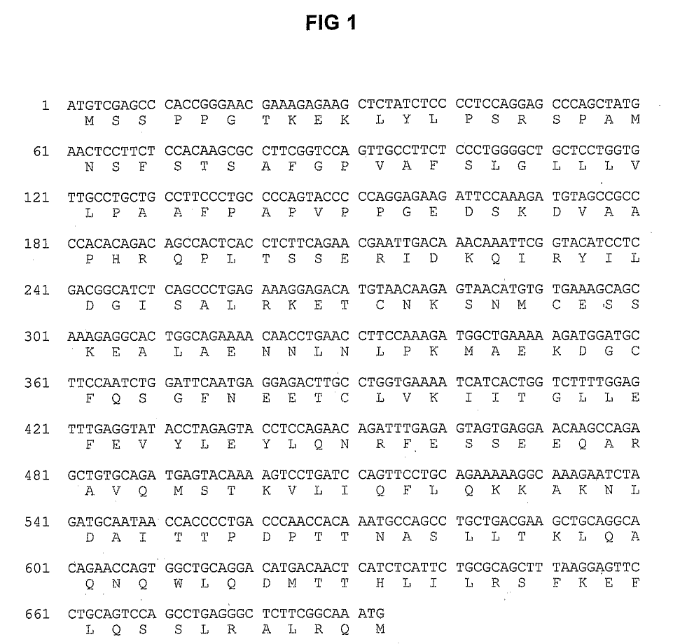

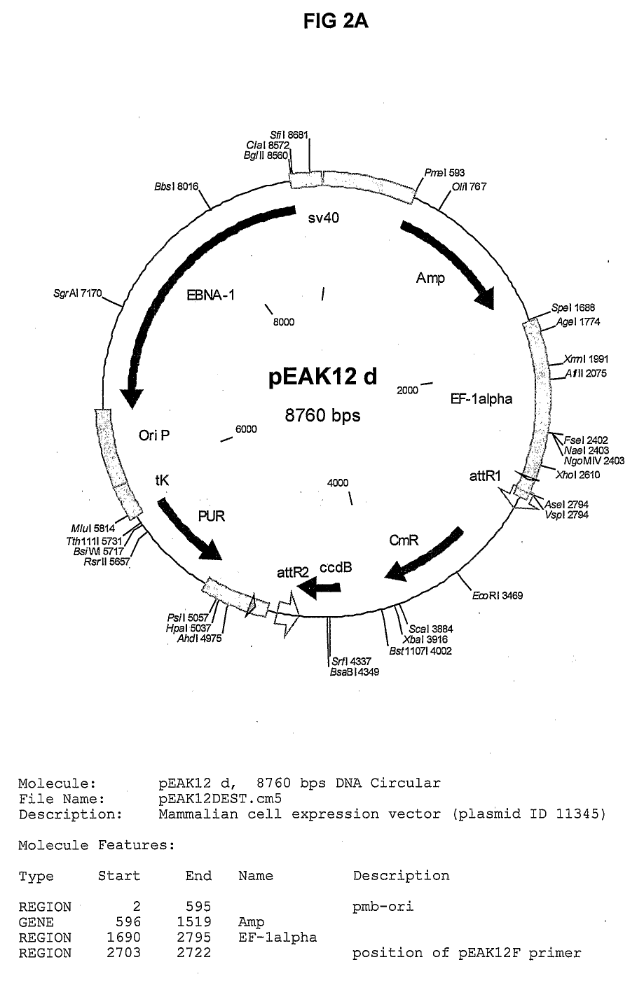

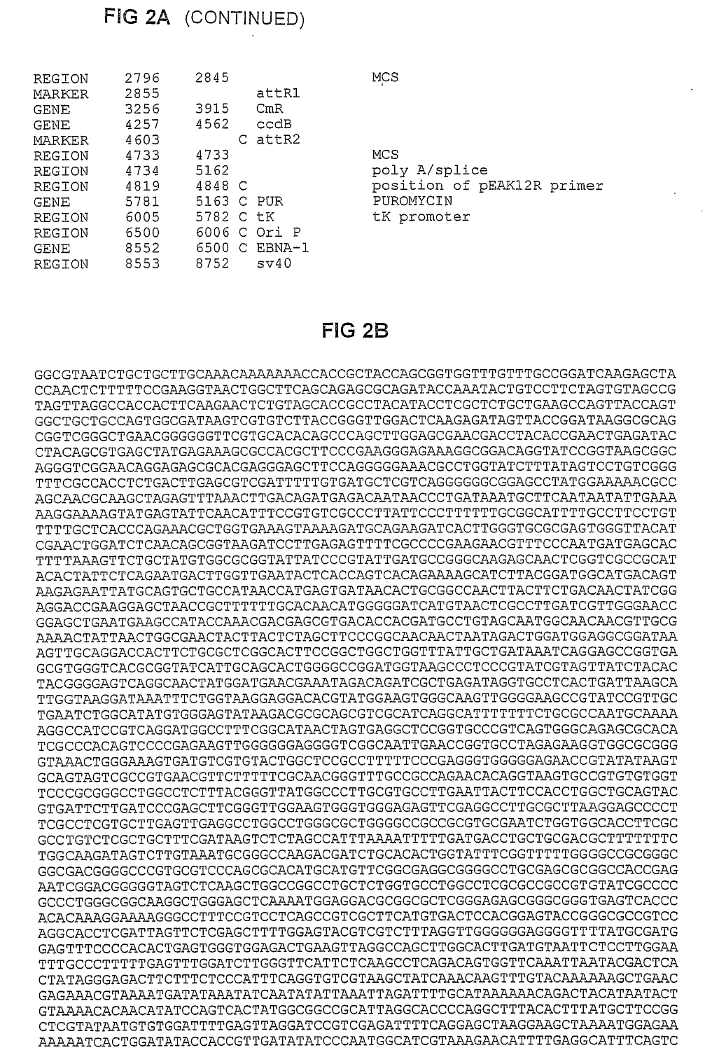

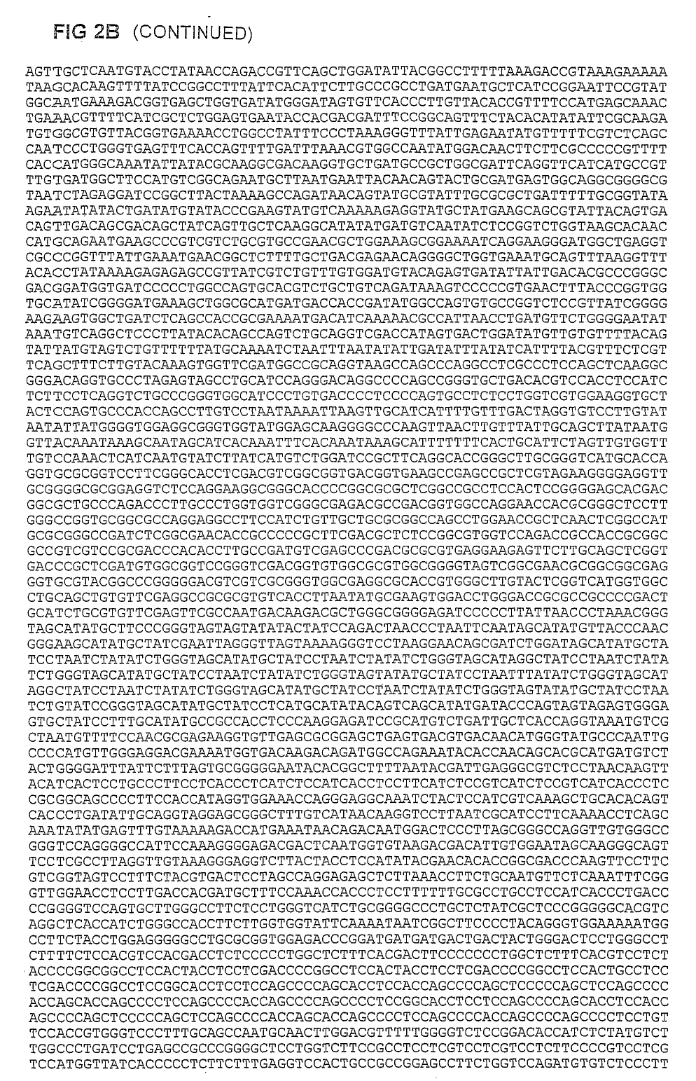

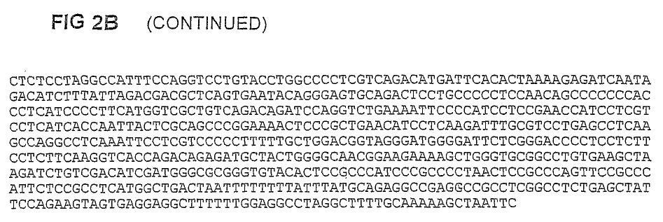

A plasmid containing the full coding sequence (ORF) of human IL-6 (FIG. 1, Seq. Id. No. 1) was purchased from Invitrogen (Invitrogen clone ID CSODIO19YPO5). The ORF was subcloned into the mammalian cell expression vector pEAK12d (FIGS. 2A and 2B) using the Gateway™ cloning methodology (Invitrogen).

1.2.1 Generation of Gateway Compatible IL-6 ORF Fused to an in Frame 6HIS Tag Sequence.

The first stage of the Gateway cloning process involves a two step PCR reaction which generates the ORF of IL-6 flanked at the 5′ end by an attB1 recombination site and Kozak sequence, and flanked at the 3′ end by a sequence encoding an in frame 6 histidine (6HIS) tag, a stop codon and the attB2 recombination site (Gateway compatible cDNA). The first PCR reaction (in a final volume of 50 μl) contains: 25 ng of CS0DI019YP05 plasmid, 2 μl dNTPs (5 mM), 5 μl of 10× Pfx polymerase buffer, 0.5 μl each of gene specific primer (100 μM) (EX1 forward and EX1 reverse) and 0.5 μl Platinum Pfx DNA polymerase (Invitrogen). The PCR reaction was performed using an initial denaturing step of 95° C. for 2 min, followed by 12 cycles of 94° C., 15 seconds and 68° C. for 30 seconds. PCR products were purified directly from the reaction mixture using the Wizard PCR prep DNA purification system (Promega) according to the manufacturer's instructions. The second PCR reaction (in a final volume of 50 μl) contained 10 μl purified PCR product, 2 μl dNTPs (5 mM), 5 μl of 10× Pfx polymerase buffer, 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 s; 45° C., 30 s and 68° C. for 3.5 min; 25 cycles of 94° C., 15 s; 55° C., 30 s and 68° C., 3.5 min. PCR products were purified as described above.

1.2.2 Subcloning of Gateway Compatible IL-6 ORF into Gateway Entry Vector pDONR201 and Expression Vector pEAK12d

The second stage of the Gateway cloning process involves subcloning of the Gateway modified PCR product into the Gateway entry vector pDONR201 (Invitrogen) as follows: 5 μl of purified PCR product is incubated with 1.5 μl pDONR201 vector (0.1 μg/μl), 2 μl BP buffer and 1.5 μof BP clonase enzyme mix (Invitrogen) at RT for 1 h. The reaction was stopped by addition of proteinase K (2 μg) and incubated at 37° C. for a further 10 min. An aliquot of this reaction (2 μl) was transformed into E. coli DH10B cells by electroporation using a Biorad Gene Pulser. Transformants were plated on LB-kanamycin plates. Plasmid mini-prep DNA was prepared from 1-4 of the resultant colonies using Wizard Plus SV Minipreps kit (Promega), and 1.5 μl of the plasmid eluate was then used in a recombination reaction containing 1.5 μl pEAK12d vector (FIG. 2) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 μl) was used to transform E. coli DH10B cells by electroporation.

Clones containing the correct insert were identified by performing colony PCR as described above except that pEAK12d primers (pEAK12d F and pEAK12d R) were used for the PCR. Plasmid mini prep DNA was isolated from clones containing the correct insert using a Qiaprep Turbo 9600 robotic system (Qiagen) or manually using a Wizard Plus SV minipreps kit (Promega) and sequence verified using the pEAK12d F and pEAK12d R primers.

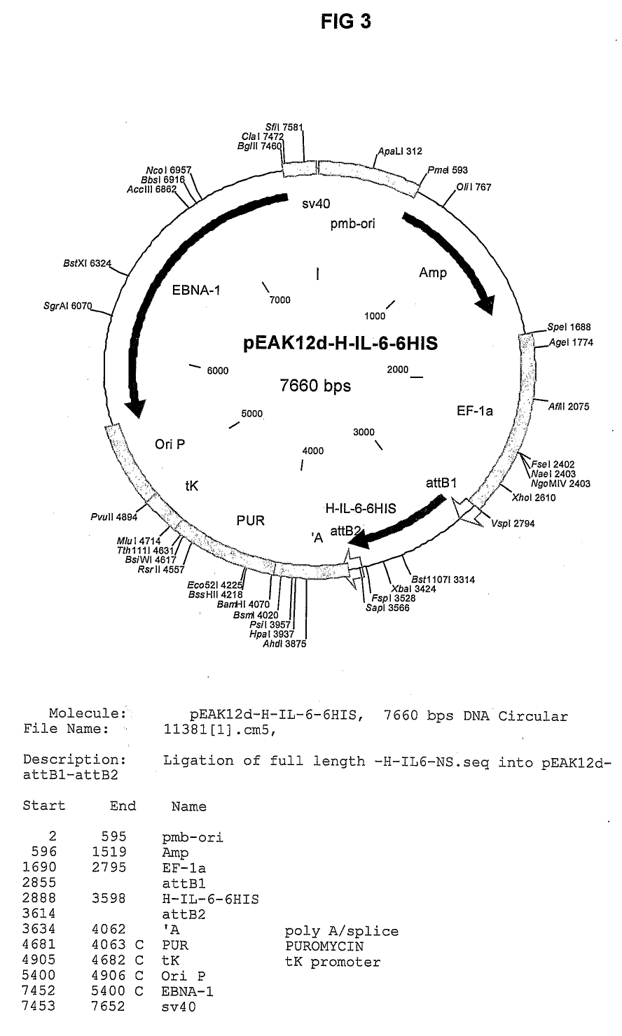

CsCl gradient purified maxi-prep DNA of plasmid pEAK12d-IL6-6HIS (plasmid ID number 11381, FIG. 3) was prepared from a 500 ml culture of sequence verified clones (Sambrook J. et al., in Molecular Cloning, a Laboratory Manual, 2^{nd} edition, 1989, Cold Spring Harbor Laboratory Press), resuspended at a concentration of 1 μg/μl in sterile water and stored at −20 C.

TABLE I
Primers for IL-6 subcloning and sequencing

| | |
|---|---|
| Primer | Sequence (5′-3′) |

| | |
|---|---|
| GCP Forward | GGGGACAAGTTTGTAC |
| | (Seq. Id. No. 12) |
| GCP Reverse | GGGGACCACTTTGTACAAGAAAGCTGGGTTTCAATGGTG |
| | ATGG |
| | (Seq. Id. No. 13) |
| EX1 Forward | GGGGACAAGTTTGTACAAAAAAGCAGGCTTCGCCACCAT |
| | GTCGAGCCC |
| | (Seq. Id. No. 14) |
| EX1 reverse | TCAATGGTGATGGTGATGGTGCATTTGCCGAAGAG |
| | (Seq. Id. No. 15) |
| pEAK12-F | GCC AGC TTG GCA CTT GAT GT |
| | (Seq. Id. No. 32) |
| pEAK12-R | GAT GGA GGT GGA CGT GTC AG |
| | (seq. Id. No. 33) |

Underlined sequence = Kozak sequence
Bold = Stop codon
Italic sequence = His tag

1.3 Cell Transfection

At the day of transfection, cells were centrifuged and re-suspended in a spinner vessel (DasGip, D) in 250 ml DMEM/F12 (1:1) medium containing 1% FBS and 4 ml/l ITS-X supplement as seeding medium (Invitrogen) at a density of 1×10⁶ cells/ml. Cells were transfected using the polyehyleneimine (PEI) method (O. Boussif et al. 1995 Proc. Natl. Acad. Sci. USA 92, pp. 7297-7301) with a ratio of 2:1 PEI:DNA. In 100 ml seeding medium 500 μg of corresponding plasmid DNA were mixed with 1 mg PEI (Polysciences, USA) and incubated for 10 min at room temperature. The mixture was added to the cell suspension and incubated for 90 minutes at 37° C. After the incubation the cell suspension was centrifuged (200×g, 10 min, 4° C.) and the cell pellet was re-suspended in 500 ml maintenance medium. Cells were incubated in a humidified atmosphere with 5% CO2 at 37° C. until encapsulation.

1.4 Cell Encapsulation

The transfected HEK293-EBNA cells obtained above and wild type HEK293-EBNA cells were encapsulated into Alginate-poly-L-Lysine-Alginate (APA) capsules using the Inotech research encapsulator (Inotech, Switzerland) which is similar to the encapsulator described in U.S. Pat. No. 6,458,296. Cells were centrifuged (200×g 10min 4° C.) and re-suspended in 2 ml washing buffer (all chemicals Inotech, Switzerland). To this suspension a 1.5% alginate solution was slowly added to yield a final cell concentration of 2.5×10⁶ cells/ml solution. The alginate-cell-suspension was taken up into a syringe (Braun Omnifit, Braun, D), which was connected to the encapsulation machine. The encapsulation was carried out using the parameters given in Table 1 using the protocol described in Table 2; all buffers were prepared according to the manufacturers' manual in sterile distilled water under sterile conditions.

At the end of final step of the encapsulation, the capsules were re-suspended in 100 ml maintenance medium and transferred into a sterile spinner vessel (Dasgip, D). The capsules were maintained in the spinner vessel incubated in a humidified atmosphere with 5% CO2 at 37° C. overnight or until injection into the animals.

The capsules had a mean diameter of 485±10 μm, as measured by microscopy.

TABLE 2
Encapsulation parameters

| | | Vibration | Vibration |
|---|---|---|---|
| Syringe Pump | Anode voltage | frequency | amplitude |
| 275 (50 ml Syringe) or | 1.16 kV | 1943 Hz | 3 |
| 456 (20 ml Syringe) | | | |

TABLE 3
Encapsulation protocole

| Solution | Time [min] | Volume [ml] |
|---|---|---|
| Polymerization | 10 | 250 |
| buffer | | |
| Poly-L-Lysine | 10 | 150 |
| Washing buffer | 1 | 150 |
| Washing buffer | 5 | 150 |
| 0.03% Alginate | 5 | 150 |
| Washing buffer | 1 | 150 |
| Depolymerization | 10 | 300 |
| buffer | | |
| Washing buffer | 1 | 150 |
| Washing buffer | 5 | 150 |
| Medium (Excell-V- | — | 100 |
| Pro) | | |

1.5. Improved Maintenance of Capsules Under Low-Shear Simulated Microgravity Conditions

Comparative experiments were performed on the maintenance of the capsules obtained above in a spinner vessel on one side, and under low-shear microgravity (10⁻¹² g) conditions using the slow turning lateral vessel STLV™ manufactured by Syntecon Inc./NASA, Houston, USA (see U.S. Pat. No. 6,730,498) on the other side. The cells were kept in maintenance medium in a humidified incubator at 37° C. with 5% CO2 in air. The capsules studied were capsules containing wild type HEK293-EBNA cells or HEK293-EBNA cells that were transiently transfected with the cDNA for h-IL-6.

It was shown that under low-shear microgravity conditions no visible capsule disruption occurred during a period of 7 days, whereas in the spinner vessel intact during the same period capsules became visibly surrounded by free-floating cells originating from disrupted capsules. For cells transfected with the cDNA for h-IL-6, h-IL-6 released in vitro was substantially lower under low-shear microgravity conditions than in the spinner vessel.

2 The Concanavalin A Model (ConA)

Materials and methods:

2.1 Animals

In all studies, male C57/BL6 mice (8 weeks of age) were used. In general, 10 animals per experimental group were used. Mice were maintained in standard conditions under a 12-hour light-dark cycle, provided irradiated food and water ad libitum.

2.1 Capsule Injection

The capsule suspension was removed from the incubator and left several minutes in the laminar flow hood to allow the capsules to sediment. The clear supernatant was removed and the concentrated capsules were taken up carefully into a syringe. 700 μl capsules were injected slowly i.p. via a 0.7 mm needle (ref 53158.01 Polylabo, Switzerland) into each mouse.

2.2 Concanavalin A Injection

Concanavalin A (ConA) was purchased from Sigma (ref. C7275, Sigma, D). ConA was i.v. injected at 18 mg/kg at 72 hours after transplantation of the capsules. Blood samples were taken at 1.30 and 8 hours after ConA injection. Measurements of cytokine and transaminase levels were performed as described below.

2.4 Readouts

2.4.1 Blood Sampling

100 μl of blood were sampled from the retro-orbital sinus at 1.30 h and 8 h after ConA injection. At the time of sacrifice,.blood was taken from the heart.

2.4.2 Detection of Cytokines and Transaminases in Blood Samples

IL-2, IL-4, IL-5, TNFβ and IFNγ cytokine levels were measured using the TH1/TH2 CBA assay (ref. 551287, Beckton Dickinson, USA). Aspartate aminotranferase (ASAT), alanine aminotransferase (ALAT), UREA blood parameters were determined using the COBAS instrument (Hitachi, Switzerland).

2.4.3 Detection of h-IL6 in Blood and Cell-Culture Supernatants.

To follow the in vivo secretion of hIL-6 from the encapsulated transfected HEK cells, blood was taken in five mice per day each day during 12 days after capsule injection. In vitro, supernatant was taken in parallel until day 10. The detection was made in serum by a commercial available ELISA kit (R&D Duoset ref. DY206). All samples were diluted ten to ten in a diluent buffer.

2.5 Results:

2.5.1 Injection of Capsules Containing Non-Transfected HEK293-EBNA Cells

In a first experiment, non-transfected encapsulated HEK293-EBNA cells were tested. It was previously shown that these capsules do not change cytokine levels or blood parameters in normal animals. The capsules were injected at day 0 and day 2. Afterwards Con A was injected. Transaminases and TNF-A levels were measured at 1.5 and 8 hours post ConA injection.

Results are illustrated in FIG. 1, which represent histograms showing the TNFα, ASAT and ALAT levels measured in blood samples of ConA treated mice that were injected with capsules containing non-tranfected HEK293-EBNA cells, and control mice.

As shown in that figure, no significant change in TNF-α or transaminases ASAT and ALAT levels could be observed in the mice that were injected with capsules containing non-tranfected HEK293-EBNA cells compared to the control mice (mice that received only a ConA injection).

2.5.2 In Vivo Activity of Capsules Containing hIL-6 Transiently Transfected HEK Cells

hIL-6 is known to decrease the levels of transaminases and TNF-α in ConA induced hepatitis in vivo (Mizuhara et al. 1994, J. Exp. Med., 179, 1529-1537).

In a first experiment, capsules containing HEK293-EBNA cells that were transfected with the cDNA coding for hIL-6 HIS were i.p or s.c. injected in C57 Bl/6 males. The hIL-6 level in the blood was measured each day just after and during 12 consecutive days after the capsule injection.

The results are illustrated in FIG. 2A, which represents a graph of the hIL-6 levels, measured in blood of mice during 12 consecutive days after i.p. or s.c. injection of capsules containing HEK293-EBNA cells that were transiently transfected with the cDNA coding for hIL-6 HIS.

As shown by that figure, hIL-6 blood levels reached peak values in between day 1 and day 5 after the i.p. capsule injection, the value of the hIL-6 blood level being always substantially higher for the mice that were i.p. injected than for those who were s.c. injected. 8 days after the i.p. or s.c. injection, the hIL-6 blood level reached a value close to the basal value.

Reproducibility: By repeating that experiment a number of times, either with the same pool of cells divided into different lots encapsulated independently as described above, or with different pools of cells prepared independently as described above, it was shown that in each case the hIL-6 blood level reached peak values in between day 1 and day 5 after the i.p. capsule injection, the hIL-6 blood level maximum value and the integrated value over the 5 first days showing no significant difference (less than 20 In a second experiment, capsules containing HEK293-EBNA cells that were transfected with the cDNA coding for hIL-6 HIS were i.p injected in C57 Bl/6 males on day 0, and on day 3. In addition 18 mg/kg ConA was i.v. injected to induce hepatitis. TNF-α and transaminase levels were measured at 1.5 and 8 hours post ConA injection.

The results are illustrated in FIGS. 2B-D which represent histograms showing the TNFα, ASAT and ALAT levels measured in blood samples of ConA treated mice that had been injected with capsules containing HEK293-EBNA cells that were transiently transfected with the cDNA coding for hIL-6 HIS, and control mice.

As shown by those figures, after ConA injection, TNF-α and transaminase levels were significantly decreased in ConA treated mice that were pre-injected with the capsules incubated in a humidified atmosphere with 5% CO₂ at 37° C., compared to ConA treated mice which received the non-transfected encapsulated HEK cells.

hIL-6 released in vivo by the capsules has thus the expected known effect of decreasing the levels of transaminases and TNF-A in ConA induced hepatitis.

2.5.3 Establishment of a Dose Response after Injection of Different Volumes of HEK293-EBNA-hIL6-HIS Capsules

An experiment to establish the dose response relationship was performed for capsules containing cells that were transiently transfected with the cDNA coding for hIL-6 HIS in order to validate the use of this technology in the ConA POC model. Systemic hIL-6 protein production was measured at day 1, day 2 and day 3 after injection of 700, 350 and 100μl of capsules.

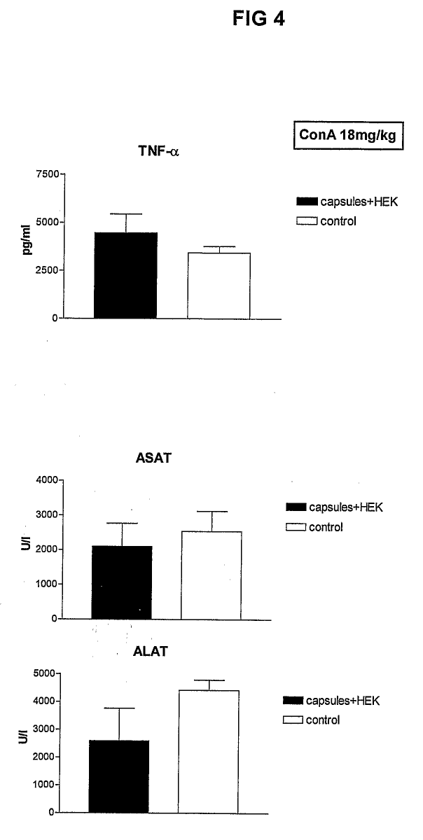

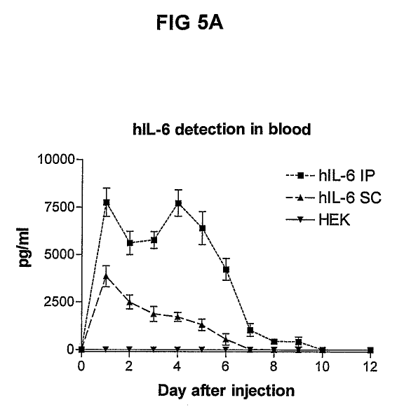

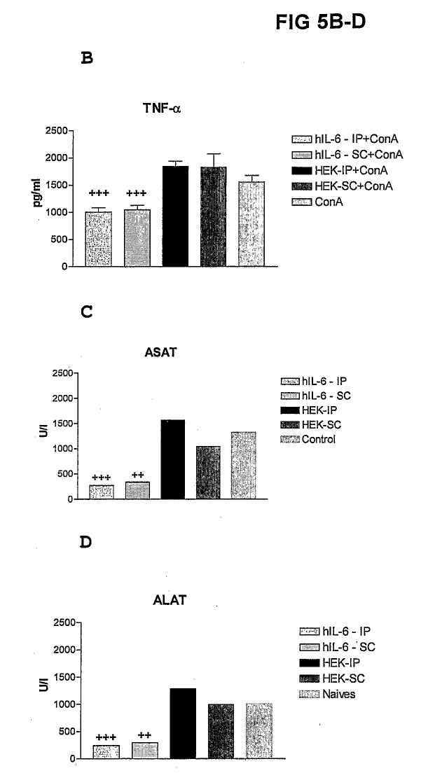

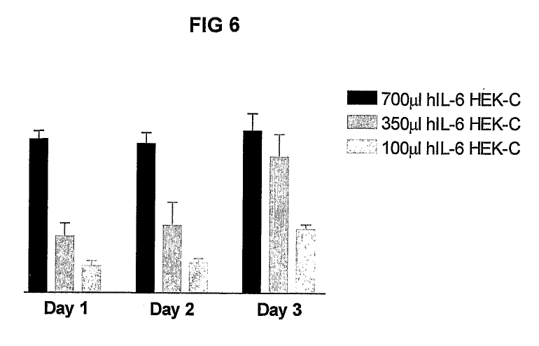

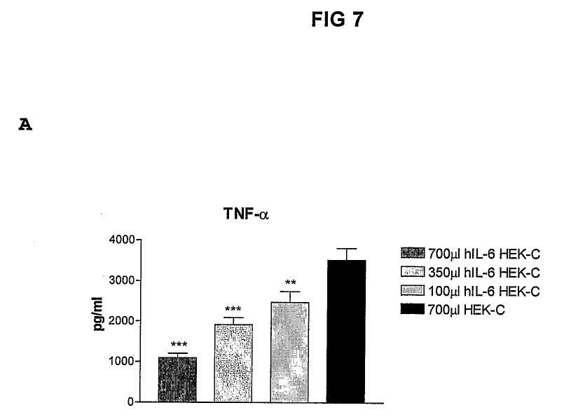

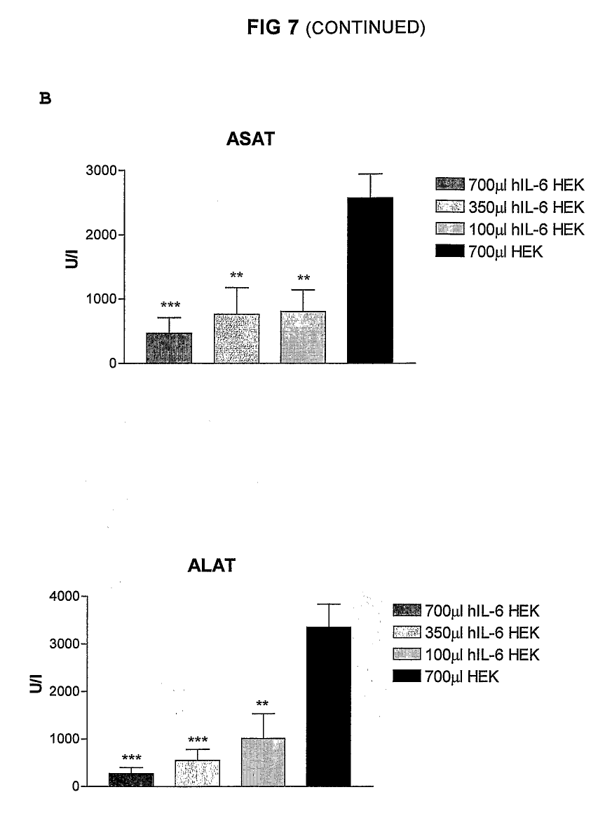

The results are illustrated in FIG. 3, which represents a histogram showing the hIL-6 levels measured in blood of mice during 3 consecutive days after i.p. injection of 700, 350 and 100 μl of capsules containing HEK293-EBNA cells that were transiently transfected with the cDNA for hIL-6-6HIS, and in FIGS. 4A-C, which represent histograms showing the TNFα, ASAT and ALAT levels measured in blood samples of ConA treated mice that were injected with 700, 350 and 100 μl, respectively, of capsules containing HEK293-EBNA cells that were transiently transfected with the cDNA for hIL-6-6HIS, and control mice.

As shown in those figures, a dose effect could be established for the production of the hIL-6 protein in blood, as well as for the biological effect on TNFα and transaminase downregulation.

### EXAMPLE 2

### Preparation of Capsules Containing Cells Transiently Transfected with a Gene for hepaCAM and Measurement of the Activity in the Mice Model of LPS-Induced TNFα release

1. Preparation of Capsules Containing Cells that are Transiently Transfected with a Gene for hepaCAM

1.1 Cell Culture and Maintenance: see Example 1

1.2 Plasmid Preparation

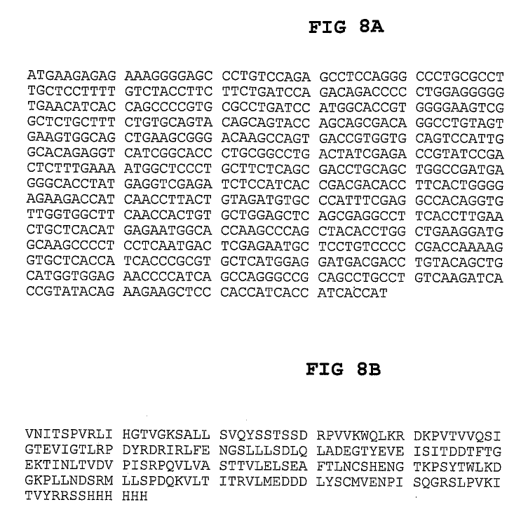

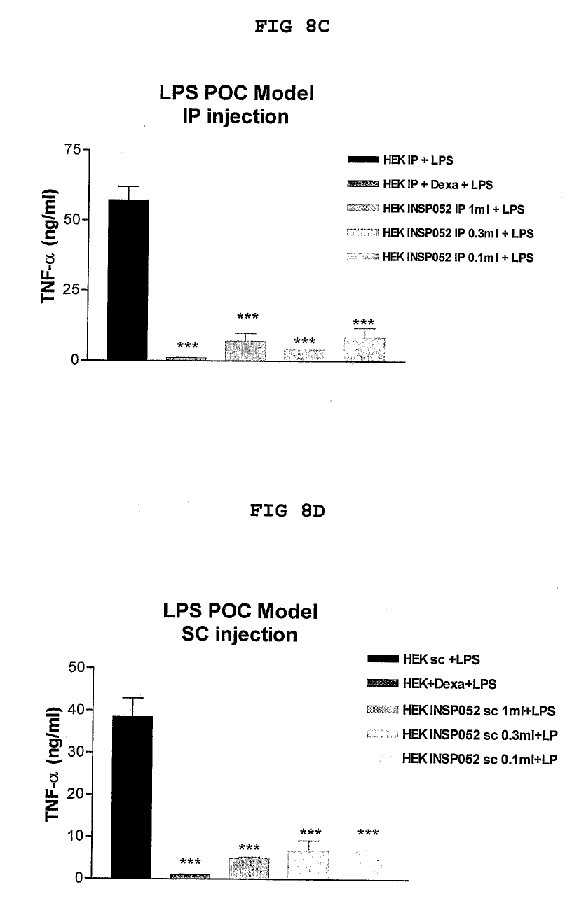

A gene for hepaCAM (see FIG. 8A for the sequence of that gene: Seq. Id. No. 7, and FIG. 8B for the sequence of that protein: Seq. Id. No. 8) was cloned into the expression vector pEAK12d as described in detail in WO 03/093316 (See also Mei Chung Moh et al. J Hepatol. June 2005; 42(6): 833-41. Epub Apr. 7, 2005 and J Biol Chem. Jul. 22, 2005; 280(29): 27366-74. Epub May 23, 2005).

Briefly that gene was first cloned into the pENTR vector of the Gateway™ cloning system (Invitrogen) using a 2-step PCR. The subcloning into the pEAK12d vector was performed according to the Gateway™ cloning manual. The gene was cloned by PCR amplification of 3 exons from the genomic sequence.

The primers used were the following

(Seq. Id. No. 20)

| | |
|---|---|
| GCAGGCTTCGCCACCATGAAGAGAGAAAGGGGAGCCC | exon 1+ PCR1 |
| TGTC | |

(Seq. Id. No. 21)

| | |
|---|---|
| TCACCCCCTCCAGGGGGTCTGTCTGGATCAGAAGAA | exon 1 |

(Seq. Id. No. 22)

| | |
|---|---|
| TTCTTCTGATCCAGACAGACCCCCTGGAGGGGGTGA | exon 2 |

(Seq. Id. No. 20)

| | |
|---|---|
| GTGGCCTCGAAATGGGCACATCTACAGTAAGGTTGA | exon 2 |

(Seq. Id. No. 24)

| | |
|---|---|
| CAACCTTACTGTAGATGTGCCCATTTCGAGGCCACA | exon 3 |

(Seq. Id. No. 25)

| | |
|---|---|
| GGAGCTTCTTCTGTATACGGTGATCTTGACAG | exon 3 |

(Seq. Id. No. 26)

| | |
|---|---|
| GTGATGGTGATGGTGGGAGCTTCTTCTGTATACGG | PCR1 |

(seq. Id. No. 18)

| | |
|---|---|
| GGGGACAAGTTTGTACAAAAAAGCAGGCTTCGCCACC | PCR2 |

(Seq. Id. No. 27)

| | |
|---|---|
| GGGGACCACTTTGTACAAGAAAGCTGGGTTTCAATGG | PCR2 |
| TGATGGTGATGGTG | |

1.3 Cell transfection: See Example 1

1.4 Cell Encapsulation: See Example 1

2. Mice Model of LPS-Induced TNFα Release the Protocol

The model of LPS-induced TNFα release in mice was set up as described in WO 98/38179.

0.3 mg/kg LPS (O111:B4, Sigma, Switzerland) was injected i.p. or s.c. into C3H/HeN mice (lots of 8 mice each) (Charles River, France) 3 days after injection of encapsulated transiently transfected HEK293-EBNA cells. Ninety minutes later blood was sampled and plasma TNFα was determined using an ELISA kit. Dexamethasone (0.1 mg/kg, sc) was solubilized in PBS and injected 15 min prior to the LPS challenge.

Results

See FIG. 8C.

Injection of LPS in animals, which had received either untransfected or hepaCAM transiently transfected encapsulated HEK293-EBNA cells by i.p. or s.c. route, showed no inhibition or significant inhibition of TNFα release, respectively. As a positive control served Dexamethasone, which inhibited LPS induced cytokine release by 80%. HepaCAM thus significantly downregulated LPS induced TNFα levels.

Those results show that hepaCAM might be useful to treat TNFα mediated inflammatory diseases.

### EXAMPLE 3

### Preparation of Capsules Containing Cells Transiently Transfected with DNA Coding for INSP114-SV2 and Measurement of the Activity in the Mice Model of LPS-Induced TNFα release

1. Preparation of Capsules Containing Cells that are Transiently Transfected with the Gene for INSP114-SV2

1.5 Cell Culture and Maintenance: See Example 1

1.6 Plasmid Preparation

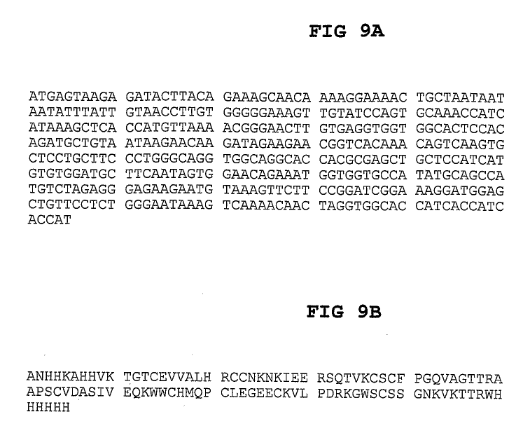

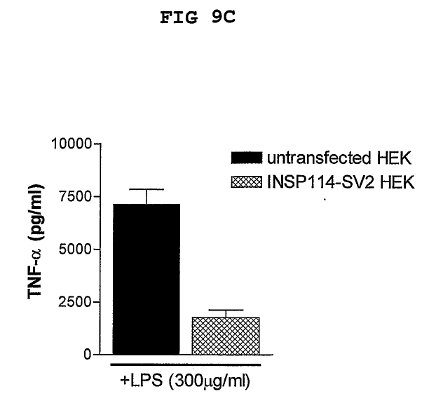

A gene for INSP114SV2 (see FIG. 9A for the sequence of that gene: Seq. Id. No. 9 and FIG. 9B for the sequence of that protein: Seq. Id. No. 10) was cloned into the expression vector pEAK12d as described in detail in WO 2004/085469.

The primers used were the following

(Seq. Id. No. 28)

| | |
|---|---|
| GCTGCAGGATGAGTAAGAGA | PCR1 |

(Seq. Id. No. 29)

| | |
|---|---|
| TCATCAGCCTTGAGGATCAC | PCR1 |

(Seq. Id. No. 30)

| | |
|---|---|
| ATGAGTAAGAGATACTTACAGAAAGC | PCR2 |

(Seq. Id. No. 31)

| | |
|---|---|
| TCACCACCTAGTTGTTTTGACTTTATTC | PCR2 |

1.7 Cell Transfection: See Example 1

1.8 Cell Encapsulation: See Example 1

2. Mice Model of LPS-Induced TNFα Release

See Example 2

Results

See FIG. 9B.

Injection of LPS in animals, which had received either untransfected or INSP114SV2 transiently transfected, encapsulated HEK293-EBNA cells by i.p. route, showed no or significant inhibition of TNFα release, respectively. As a positive control served Dexamethasone, which inhibited LPS induced cytokine release by 80%. INSP114SV2 thus significantly downregulated LPS induced TNFα levels.

Those results show that INSP114SV2 might be useful to treat TNFα mediated inflammatory diseases.

### EXAMPLE 4

### Preparation of Capsules Containing Cells Transiently Transfected with a Gene for EPO and Measurement of the EPO Concentration in Blood and the Hematocrit in a Mice Model

1. Preparation of Capsules Containing Cells that are Transiently Transfected with a Gene for EPO

1.1 Cell Culture and Maintenance: (See Example 1)

1.2 Plasmid Preparation

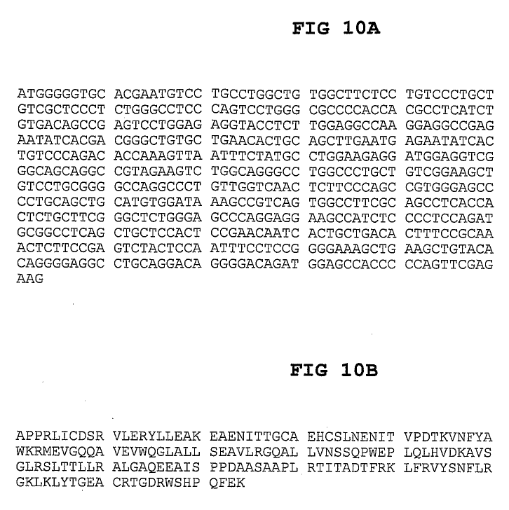

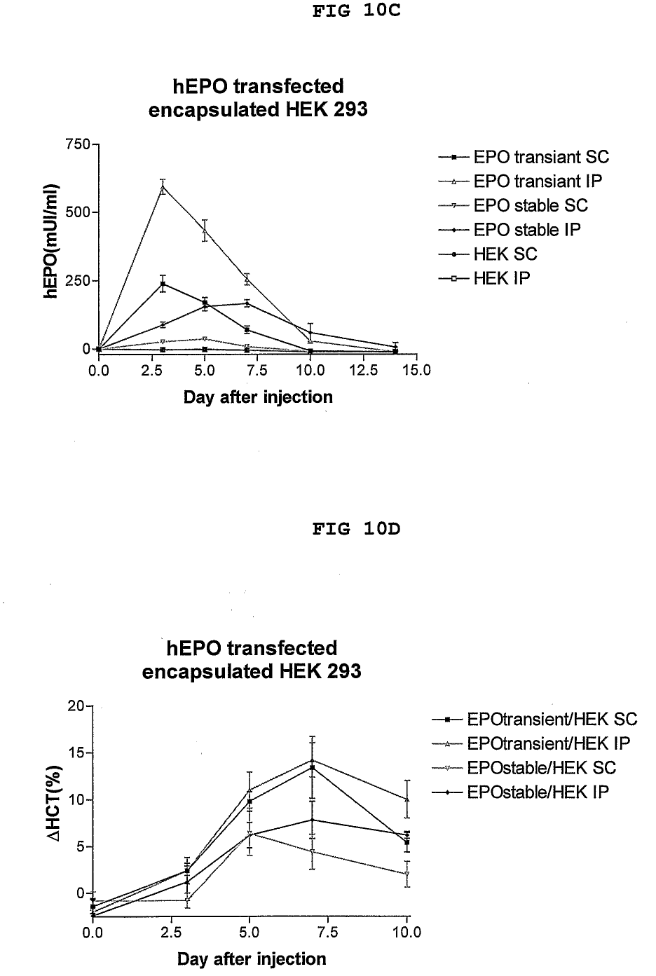

A gene for EPO (see FIG. 10A for the sequence of that gene: Seq. Id. No. 5 and FIG. 10B for the sequence of that protein: Seq. Id. No. 6) was cloned into the expression vector pEAK12d using a protocol similar to that described in Example 1 for a gene for IL-6, and the following primers

(Seq. Id. No. 16)

| | |
|---|---|
| CTGGGGGTGGCTCCATCTGTCCCCTGTCCTGC | PCR1 |

(Seq. Id. No. 17)

| | |
|---|---|
| GCAGGCTTCGCCACCATGGGGGTGCACGAATGTCC | PCR1 |

(Seq. Id. No. 18)

| | |
|---|---|
| GGGGACAAGTTTGTACAAAAAAGCAGGCTTCGCCACC | PCR2 |

(Seq. Id. No. 19)

| | |
|---|---|
| GGGGACCACTTTGTACAAGAAAGCTGGGTTTCACTTCTCGAACTG | PCR2 |
| GGGGTGGCTCCA | |

1.3 Cell Transfection

1.3.1: Preparation of Cells Transiently Transfected with the Gene for EPO

At the day of transfection, HEK293-EBNA cells were centrifuged and re-suspended in a spinner vessel (DasGip, D) in 250 ml DMEM/F12 (1:1) medium containing 1% FBS and 4 ml/l ITS-X supplement as seeding medium (Invitrogen) at a density of 1×10⁶ cells/ml. Cells were transfected using the polyehyleneimine (PEI) method (O. Boussif et al. 1995 Proc. Natl. Acad. Sci. USA 92, pp. 7297-7301) with a ratio of 2:1 PEI:DNA. In 100 ml seeding medium 500 μg of corresponding plasmid DNA were mixed with 1 mg PEI (Polysciences, USA) and incubated for 10 min at room temperature. The mixture was added to the cell suspension and incubated for 90 minutes at 37° C. After the incubation the cell suspension was centrifuged (200×g, 10 min, 4° C.) and the cell pellet was re-suspended in 500 ml maintenance medium. Cells were incubated in a humidified atmosphere with 5% CO₂ at 37° C. until encapsulation.

1.3.2: Preparation of Semi-Stable Pools of Cells Transfected with the Gene for EPO

The technique is based on an episomal replication of the gene for EPO inserted in pEAK12d, which includes in its backbone the puromycin-N-acetyl-transferase resistance gene, and application of purimycin selection pressure to select semi-stable pools of cells.

Initial Transfection of the EPO Gene

HEK293-EBNA cells were maintained in suspension in the Ex-cell VPRO serum-free medium (seed stock, maintenance medium, JRH). On the day of transfection, cells were counted, centrifuged (low speed) and the pellet re-suspended into the desired volume of transfection medium, i.e. DMEM/F12 (1:1) (FEME medium, Invitrogen) supplemented with 1% FCS (JRH) and 4ml/l Insulin Transferrin Selenium (Gibco) to yield a cell concentration of 1XE6 viable cells/ml. The DNA stock obtained from the cloning of the gene for EPO into the PEAK12d in 1.2 above (1 mg/ml stock) was diluted at 2mg/transfection liter volume (co-transfected with 2% eGFP reporter gene) in FEME medium. The PolyEthyleneImine transfection agent (PEI, 4 mg/liter volume, Polysciences) was then added to the cDNA solution, vigorously vortexed (30 seconds) and incubated at room temperature for 10 minutes (generating the “transfection Mix”).

This transfection mix was then added to the spinner and incubated for 90 minutes in a CO2 incubator (5% CO₂ and 37° C.). After the 90 minutes period, allowing for the PEI-EPO cDNA complexes to be incorporated into the HEK293-EBNA cells, cells were centrifuged and re-suspended into fresh Ex-cell VPRO serum-free medium such as to be kept as a single cell suspension. Then, 24 to 48 hours after transfection, a sample of the above-mentioned EPO HEK293-EBNA cells growing in serum free medium was inspected under a fluorescent microscope such as to visualize the reporter gene having rendered transfected cells fluorescent. Only if fluorescence was seen on more than 80% of the cells was this culture kept for further semi-stable pressure selection.

Puromycin Double Pressure Selection

After 72 hours post-transfection, the medium of the suspension-growing HEK293-EBNA cells expressing EPO was exchanged with fresh Ex-cell VPRO medium containing 7.5 μg/ml of Puromycin (Clontech) and this puromycin pressure was maintained for 6 days. During this period, the pressure medium was changed with fresh pressure medium (still containing puromycin) when lactate concentrations reached more than 1.3 g/l (growth inhibition due to lactate). Non-recombinant cells died (only 10% of cells as PEI transfection efficiency is high, above 85%) and EPO-recombinant cells survived the pressure selection as visualized by fluorescent microscopy and suspension cells growing still (population doubling).

At the end of the selection period, the medium was exchanged with normal growth medium (without puromycin) and cells monitored for protein expression for two or three weeks. A second round of puromycin selection pressure (identical procedure as above) was applied to the cell suspension for another 6 days such as to make sure that all non-recombinant cells had been eliminated.

Cell growth was monitored, a small cell stock was laid down by cryopreservation of part of the culture, whilst maintaining enough cells in culture such as to be able to harvest cells for the encapsulation procedure.

The semi-stable cells obtained can express the EPO gene for about three months.

1.4 Cell Encapsulation

The transiently transfected cells obtained in 1.3.1 and the semi-stable cells obtained in 1.3.2 were encapsulated as described in Example 1 in 1.4.

2. Measurement of the EPO Concentration in Blood and the Hematocrit in a Mice Model

2.1 Protocol

In all studies, male C57/BL6 mice (8 weeks of age) were used. In general, 5 animals per experimental group were used. Mice were maintained in standard conditions under a 12-hour light-dark cycle, provided irradiated food and water ad libitum.

The capsule suspension was removed from the incubator and left several minutes in the laminar flow hood to allow the capsules to sediment. The clear supernatant was removed and the concentrated capsules were taken up carefully into a syringe. 700 μl capsules containing the EPO transiently transfected cells, the EPO transfected semi-stable cells or non-transfected cells were injected slowly i.p. or s.c. via a 0.7 mm needle (ref 53158.01 Polylabo, Switzerland) into each mouse.

The EPO blood level was followed during 10 consecutive days after capsule injection using a hEPO ELISA kit from R&D Systems (Quantitin IVD Human EPO, catalog No. DEP00). The hematocrit was determined.

2.2 Results

As apparent from FIGS. 10C and 10D, an increase in EPO concentration in blood (10C) corresponding to an increase in hematocrit (10D) was measured during the 14 days of the experiment in the mice which had obtained (i.p. or s.c. route) encapsulated cells that were either transiently or stably transfected. No such effect was observed in the control animals that had obtained untransfected encapsulated cells. Remarkably and surprisingly, the EPO concentration and the hematocrit in mice that had obtained transiently transfected encapsulated cells reached higher levels than in the mice that had obtained stably transfected encapsulated cells.

### EXAMPLE 5

### Preparation of Capsules Containing Cells Transiently Transfected with a Gene for mIL-18BP and Measurement of the mIL-18BP Blood Level in a Mice Model

1. Preparation of Capsules Containing Cells that are Transiently Transfected with a Gene for mIL-18BP

1.1 Cell Culture and Maintenance: See Example 1

1.2 Plasmid Preparation

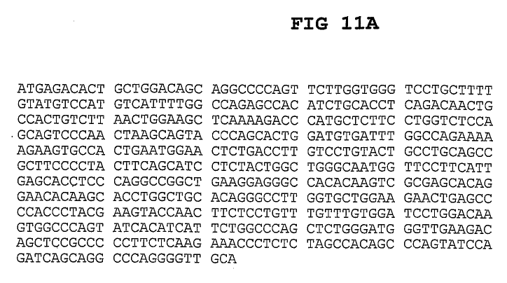

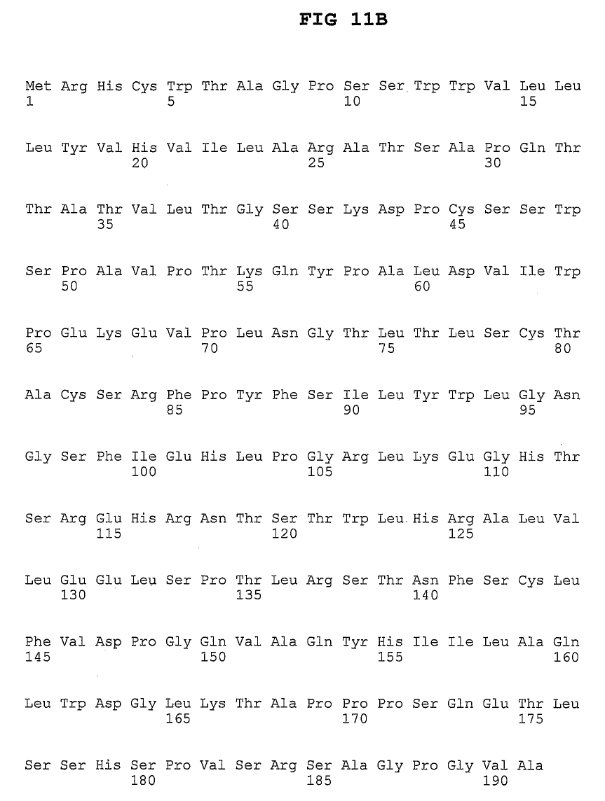

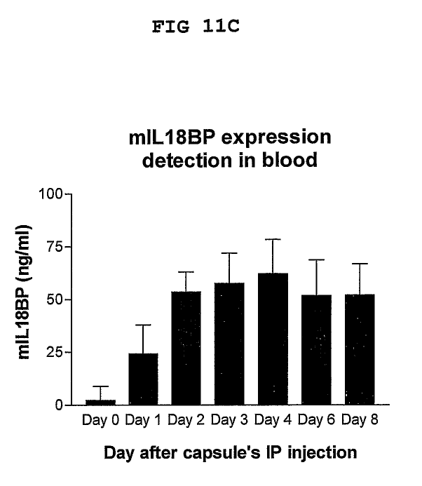

A gene for mIL-18BP (see FIG. 11A for the sequence of that gene: Seq. Id. No. 3 and FIG. 11B for the sequence of that protein: Seq. Id. No. 4) was cloned into the expression vector pEAK12d as described in detail in Mallat Z. et al. 2002 Circ. Res. 91, pp.441-448.

Briefly the plasmid pCEP4-mIL18BP-d 23 was used as a template for the amplification reaction. The PCR product was digested with NotI+HinDIII, then purified by Nucleospin™ spin column kit (Macherey-Nagel) and ligated into an HinDIII+NotI digested pEAK8 vector (Edge BioSystems, Gaithersburg, Md., USA).

1.3 Cell Transfection: See Example 1

1.4 Cell Encapsulation: See Example 1

2. Measurement of the mIL-18BP Blood Level in a Mice Model

2.1 Protocol

A group of 17 male C57/BL6 mice (8 weeks of age) was used. Mice were maintained in standard conditions under a 12-hour light-dark cycle, provided irradiated food and water ad libitum.

The capsule suspension was removed from the incubator and left several minutes in the laminar flow hood to allow the capsules to sediment. The clear supernatant was removed and the concentrated capsules were taken up carefully into a syringe. 700 μl capsules containing the mIL-18BP transiently transfected cells were injected slowly i.p. via a 0.7 mm needle (ref 53158.01 Polylabo, Switzerland) into each mouse. The EPO blood level was followed during 8 consecutive days after capsule injection using a laboratory made ELISA for m-IL18BP.

The procedure for that ELISA was the following.

The plate Labsystem combiplate 12EB was used. The coating was: 5 mg/ml in 0.1 ml PBS1X anti-murine IL-18BP. Antigen affinity purified polyclonal antibody from rabbit sera. Incubation was: O/N 4° C. followed by washing with: PBS 1X+0.05% Tween 20, blocking with: PBS 1X BSAO.2% 1 hour at 37° C. (BSA Sigma ref. A-2153), and washing with: PBS 1X+0.05% Tween 20. The standard was: mIL-18BP 300 ng/ml to 0.1 ng/ml, samples: 0.1 ml 2 hours 37° C. in PBS 1X, BSA 0.1%, Tween 20 0.05%. Washing was carried out with: PBS 1X+0.05% Tween 20. 0.1 ml biotinylated anti-murine IL18BP (Peprotech) 0.3 mg/ml was added during 2 hours at 37° C. Washing was carried out with: PBS 1X +0.05% Tween 20, and 0.1 ml extravidin HRP (Sigma ref. E2886) 1/5000 was added gently during 30 minutes at room temperature. The reaction was stopped with 0.05 ml H2SO4 20%. Reading was made at 492 nm.

2.2 Results

As apparent from FIG. 11C, m-IL18BP blood levels reached a peak value between day 3 and day 6 after the i.p. capsule injection, the m-IL18BP level after 8 days remaining very high.

1-26. (canceled)

27. A composition of matter comprising a capsule containing cells that are transiently transfected with a gene of interest and entrapped within a biocompatible polymer membrane.

28. The composition of matter according to claim 27, wherein the cells are animal cells.

29. The composition of matter according to claim 28, wherein said animal cells are mammalian.

30. The composition of matter according to claim 27, wherein the gene of interest is fused to a signal sequence for secretion of the protein, inserted into an expression cassette or inserted into a plasmid.

31. The composition of matter according to claim 27, wherein the biocompatible polymer membrane is composed of alginate-poly-L-lysine-alginate (APA).

32. The composition of matter according to claim 27, wherein the biocompatible polymer membrane has pores with a cut-off size of 90 kDa to 30 kDa or 80 kDa to 60 kDa.

33. The composition of matter according to claim 31, wherein the biocompatible polymer membrane has pores with a cut-off size of 90 kDa to 30 kDa or 80 kDa to 60 kDa.

34. The composition of matter according to claim 27, wherein the capsule has a mean diameter of 100 to 1500 μm, 250 to 600 μm or 440 to 530 μm.

35. The composition of matter according to claim 27, wherein the capsule has been maintained under low-shear microgravity conditions.

36. The composition of matter according to claim 27, further comprising one or more pharmaceutically acceptable carriers, diluents or excipients.

37. The composition of matter according to claim 27, wherein the gene of interest is coding for an antigen/immunogen and/or an adjuvant.

38. The composition of matter according to claim 37, wherein the antigen/immunogen is a bacterial, viral, fungal, parasitic or tumor antigen.

39. The composition of matter according to claim 27, wherein the capsule comprises at least two types of cells, each transfected with a gene encoding one or more antigen and/or one or more adjuvant and wherein the two genes are not identical.

40. A method of preparing a capsule comprising the steps of transiently transfecting cells with a gene of interest and encapsulating the transiently transfected cells.

41. The method according to claim 40, further comprising maintaining the capsule under low-shear microgravity gravity conditions.

42. A method for assessing an in vivo activity of the protein expressed and secreted by a gene of interest, which comprises administering a capsule according to claim 27 to a multicellular organism and detecting an activity of said protein.

43. The method according to claim 42, wherein the multicellular organism is a mammal.

44. The method according to claim 43, wherein the mammal is selected from the group consisting of mouse, rats, dogs, goats, sheep, cows and monkeys.

45. The method according to claim 43, wherein administration of the capsule is performed by i.p. injection of a mammal.

46. The method according to claim 43, wherein the in vivo activity to be detected is completely induced within a period of 3 to 14 days, or within a period of 3 to 5 days after administration of said protein to said mammal.

47. The method according to claim 46, wherein said mammal is a mouse with Concanavalin A (ConA) induced liver toxicity.

48. A method of administering a protein of interest to a subject comprising administering a composition of matter comprising a capsule according to claim 27 that expresses a protein of interest to a subject.

49. The method according to claim 48, wherein the protein of interest is an antigen for immunization or vaccination.

50. The method according to claim 48, wherein the subject is selected from the group consisting of humans; animals kept for research purposes; livestock; and companion animals.

51. A kit comprising a composition of matter according to claim 27 and means for the application of said composition of matter to a subject.


SEQUENCE LISTING (33 sequences)

SEQ ID NO: 1 - DNA, 693 bp, Homo sapiens
  misc_feature: IL-6
  sig_peptide (58)..(144)
  CDS (58)..(693)
atgtcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatg

SEQ ID NO: 2 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 3 - DNA, 573 bp, Mus musculus
  misc_feature: mIL-18 BP
  sig_peptide (1)..(78)
  CDS (1)..(573)
atgagacactgctggacagcaggccccagttcttggtgggtcctgcttttgtatgtccatgtcattttggccagagccacatctgcacctcagacaactgccactgtcttaactggaagctcaaaagacccatgctcttcctggtctccagcagtcccaactaagcagtacccagcactggatgtgatttggccagaaaaagaagtgccactgaatggaactctgaccttgtcctgtactgcctgcagccgcttcccctacttcagcatcctctactggctgggcaatggttccttcattgagcacctcccaggccggctgaaggagggccacacaagtcgcgagcacaggaacacaagcacctggctgcacagggccttggtgctggaagaactgagccccaccctacgaagtaccaacttctcctgtttgtttgtggatcctggacaagtggcccagtatcacatcattctggcccagctctgggatgggttgaagacagctccgcccccttctcaagaaaccctctctagccacagcccagtatccagatcagcaggcccaggggttgca

SEQ ID NO: 4 - protein, 191 aa, Mus musculus
MRHCWTAGPSSWWVLLLYVHVILARATSAPQTTATVLTGSSKDPCSSWSPAVPTKQYPALDVIWPEKEVPLNGTLTLSCTACSRFPYFSILYWLGNGSFIEHLPGRLKEGHTSREHRNTSTWLHRALVLEELSPTLRSTNFSCLFVDPGQVAQYHIILAQLWDGLKTAPPPSQETLSSHSPVSRSAGPGVA

SEQ ID NO: 5 - DNA, 603 bp, Homo sapiens
  misc_feature: Erythropoietin (EPO)
  sig_peptide (1)..(81)
  CDS (1)..(603)
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccaggaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatggagccacccccagttcgagaag

SEQ ID NO: 6 - protein, 201 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQEEAISPPDAASAAPLRTITADTFRKLFRVYSNFLRGKLKLYTGEACRTGDRWSHPQFEK

SEQ ID NO: 7 - DNA, 738 bp, Homo sapiens
  misc_feature: hepaCAM
  sig_peptide (1)..(99)
  CDS (1)..(738)
atgaagagagaaaggggagccctgtccagagcctccagggccctgcgccttgctccttttgtctaccttcttctgatccagacagaccccctggagggggtgaacatcaccagccccgtgcgcctgatccatggcaccgtggggaagtcggctctgctttctgtgcagtacagcagtaccagcagcgacaggcctgtagtgaagtggcagctgaagcgggacaagccagtgaccgtggtgcagtccattggcacagaggtcatcggcaccctgcggcctgactatcgagaccgtatccgactctttgaaaatggctccctgcttctcagcgacctgcagctggccgatgagggcacctatgaggtcgagatctccatcaccgacgacaccttcactggggagaagaccatcaaccttactgtagatgtgcccatttcgaggccacaggtgttggtggcttcaaccactgtgctggagctcagcgaggccttcaccttgaactgctcacatgagaatggcaccaagcccagctacacctggctgaaggatggcaagcccctcctcaatgactcgagaatgctcctgtcccccgaccaaaaggtgctcaccatcacccgcgtgctcatggaggatgacgacctgtacagctgcatggtggagaaccccatcagccagggccgcagcctgcctgtcaagatcaccgtatacagaagaagctcccaccatcaccatcaccat

SEQ ID NO: 8 - protein, 246 aa, Homo sapiens
MKRERGALSRASRALRLAPFVYLLLIQTDPLEGVNITSPVRLIHGTVGKSALLSVQYSSTSSDRPVVKWQLKRDKPVTVVQSIGTEVIGTLRPDYRDRIRLFENGSLLLSDLQLADEGTYEVEISITDDTFTGEKTINLTVDVPISRPQVLVASTTVLELSEAFTLNCSHENGTKPSYTWLKDGKPLLNDSRMLLSPDQKVLTITRVLMEDDDLYSCMVENPISQGRSLPVKITVYRRSSHHHHHH

SEQ ID NO: 9 - DNA, 405 bp, Homo sapiens
  misc_feature: INSP114V2
  sig_peptide (1)..(90)
  CDS (1)..(405)
atgagtaagagatacttacagaaagcaacaaaaggaaaactgctaataataatatttattgtaaccttgtgggggaaagttgtatccagtgcaaaccatcataaagctcaccatgttaaaacgggaacttgtgaggtggtggcactccacagatgctgtaataagaacaagatagaagaacggtcacaaacagtcaagtgctcctgcttccctgggcaggtggcaggcaccacgcgagctgctccatcatgtgtggatgcttcaatagtggaacagaaatggtggtgccatatgcagccatgtctagagggagaagaatgtaaagttcttccggatcggaaaggatggagctgttcctctgggaataaagtcaaaacaactaggtggcaccatcaccatcaccat

SEQ ID NO: 10 - protein, 135 aa, Homo sapiens
MSKRYLQKATKGKLLIIIFIVTLWGKVVSSANHHKAHHVKTGTCEVVALHRCCNKNKIEERSQTVKCSCFPGQVAGTTRAAPSCVDASIVEQKWWCHMQPCLEGEECKVLPDRKGWSCSSGNKVKTTRWHHHHHH

SEQ ID NO: 11 - DNA, 8481 bp, Artificial
  pEAK12d vector
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgcatgttcggcgaggcggggcctgcgagcgcggccaccgagaatcggacgggggtagtctcaagctggccggcctgctctggtgcctggcctcgcgccgccgtgtatcgccccgccctgggcggcaaggctgggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttgggttcattctcaagcctcagacagtggttcaaattaatacgactcactatagggagacttctttctcccatttcaggtgtcgtaagctatcaaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcaggcggggcgtaatctagaggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtcccccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttcgatggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatccgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccggcacctcctccagccccagcacctccaccagccccagctcccccagctccagccccaccagcaccagcccctccagccccaccagccccagcccctccggcacctcctccagccccagcacctccaccagccccagctcccccagctccagccccaccagcaccagcccctccagccccaccagccccagcccctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaagctaagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

SEQ ID NO: 12 - DNA, 16 bp, Artificial
  Primer IL-6 GCP Forward
ggggacaagtttgtac

SEQ ID NO: 13 - DNA, 43 bp, Artificial
  Primer IL-6 GCP Reverse
ggggaccactttgtacaagaaagctgggtttcaatggtgatgg

SEQ ID NO: 14 - DNA, 48 bp, Artificial
  Primer IL-6 EX1 Forward
ggggacaagtttgtacaaaaaagcaggcttcgccaccatgtcgagccc

SEQ ID NO: 15 - DNA, 35 bp, Artificial
  Primer IL-6 EX1 Reverse
tcaatggtgatggtgatggtgcatttgccgaagag

SEQ ID NO: 16 - DNA, 32 bp, Artificial
  Primer EPO-6HISp PCR1
ctgggggtggctccatctgtcccctgtcctgc

SEQ ID NO: 17 - DNA, 35 bp, Artificial
  Primer EPO-B1p PCR1
gcaggcttcgccaccatgggggtgcacgaatgtcc

SEQ ID NO: 18 - DNA, 37 bp, Artificial
  Primer attB1-K PCR2
ggggacaagtttgtacaaaaaagcaggcttcgccacc

SEQ ID NO: 19 - DNA, 57 bp, Artificial
  Primer attB2-STOP-SII PCR2
ggggaccactttgtacaagaaagctgggtttcacttctcgaactgggggtggctcca

SEQ ID NO: 20 - DNA, 41 bp, Artificial
  Primer hepaCAM 052-B1p-K-1-F Exon 1 + PCR1
gcaggcttcgccaccatgaagagagaaaggggagccctgtc

SEQ ID NO: 21 - DNA, 36 bp, Artificial
  Primer hepaCAM 052-103-R Exon 1
tcaccccctccagggggtctgtctggatcagaagaa

SEQ ID NO: 22 - DNA, 36 bp, Artificial
  Primer hepaCAM 052-68-F Exon 2
ttcttctgatccagacagaccccctggagggggtga

SEQ ID NO: 23 - DNA, 36 bp, Artificial
  Primer hepaCAM 052-445-R Exon 2
gtggcctcgaaatgggcacatctacagtaaggttga

SEQ ID NO: 24 - DNA, 36 bp, Artificial
  Primer hepaCAM 052-411-F Exon 3
caaccttactgtagatgtgcccatttcgaggccaca

SEQ ID NO: 25 - DNA, 32 bp, Artificial
  Primer hepaCAM 052-720-R Exon 3
ggagcttcttctgtatacggtgatcttgacag

SEQ ID NO: 26 - DNA, 35 bp, Artificial
  Primer hepaCAM 5His-052-720-R PCR1
gtgatggtgatggtgggagcttcttctgtatacgg

SEQ ID NO: 27 - DNA, 51 bp, Artificial
  Primer hepaCAM B2-stop-His6-R PCR2
ggggaccactttgtacaagaaagctgggtttcaatggtgatggtgatggtg

SEQ ID NO: 28 - DNA, 20 bp, Artificial
  Primer INSP114-F1 PCR1
gctgcaggatgagtaagaga

SEQ ID NO: 29 - DNA, 20 bp, Artificial
  Primer INSP114-R1 PCR1
tcatcagccttgaggatcac

SEQ ID NO: 30 - DNA, 26 bp, Artificial
  Primer INSP114-F2 PCR2
atgagtaagagatacttacagaaagc

SEQ ID NO: 31 - DNA, 28 bp, Artificial
  Primer INSP114-R2 PCR2
tcaccacctagttgttttgactttattc

SEQ ID NO: 32 - DNA, 20 bp, Artificial
  Primer pEAK12-F
gccagcttggcacttgatgt

SEQ ID NO: 33 - DNA, 20 bp, Artificial
  Primer pEAK12-R
gatggaggtggacgtgtcag